(19) United States
(12) Patent Application Publication
(10) Pub. No.: US 2010/0297623 A1
(43) Pub. Date: Nov. 25, 2010

(54) NOVEL HUMAN ssDNA BINDING PROTEINS AND METHODS OF CANCER DIAGNOSIS

(75) Inventors: Kum Kum Khanna, Herston (AU); Derek Richard, Ferny Grove (AU); Malcolm F. White, Fife (GB)

Correspondence Address: SHERIDAN ROSS PC, 1560 BROADWAY, SUITE 1200, DENVER, CO, 80202, US

(73) Assignee: THE COUNCIL OF THE QUEENSLAND INSTITUTE OF MEDICAL RESEARCH, Herston (AU)

(21) Appl. No.: 12/530,085

(22) PCT Filed: Mar. 7, 2008
(86) PCT No.: PCT/AU08/00181

§ 371 Date: Feb. 19, 2010

(30) Foreign Application Priority Data
Mar. 7, 2007 (AU) 2007901166

(51) Int. Cl. C12Q 1/68; G01N 33/53; C12Q 1/02; C07K 14/435; C07K 16/18; C07H 21/04

(52) U.S. Cl. 435/6; 435/7.1; 435/29; 530/350; 530/387.9; 536/23.5

(57) ABSTRACT

A method for detecting transformed cells or tumour cells, a method for diagnosing or prognosing cancer or for assessing a predisposition to cancer, and kits for use in the methods are disclosed. The methods particularly involve the detection of overexpression of an ssDNA binding protein (SSB) or polypeptide comprising the following amino acid sequence: FX¹X²DX³KPGLKNLNX⁴X⁵FIVLEX⁶GRVTKTKDGHEVRX⁷CKVADKTGSIX⁸ISVWDX⁹X¹⁰GX¹¹LIQPGDI IRLTX¹²GYASX¹³X¹⁴KGCLTLYTGRGGX¹⁵LQKIGEFCMVYSEVPNFSEPNPX¹⁶YX¹⁷ X¹⁸QQ (SEQ ID NO: 1).

### FIELD OF THE INVENTION

The present invention relates to a method for detecting transformed cells or tumour cells, a method for diagnosing or prognosing cancer or for assessing a predisposition to cancer, and kits for use in said methods. More particularly, the invention relates to methods involving the detection of overexpression of a human SSB protein or polypeptide, and kits for use in said methods.

### BACKGROUND OF THE INVENTION

DNA exists predominantly in a duplex form that is preserved via specific base pairing. This affords a considerable degree of protection against chemical or physical damage thereby preserving its coding potential. However, there are many situations, either due to DNA damage or during programmed cellular processes such as DNA replication and transcription, when the DNA duplex is separated into two single-stranded DNA (ssDNA) strands. It is very important to control the generation of ssDNA and protect it when formed, and for this reason all cellular organisms and many viruses encode protective ssDNA binding proteins (SSBs).

SSBs are ubiquitous and essential for a wide variety of cellular processes including DNA replication, recombination, DNA damage detection and repair. SSBs have multiple roles in binding and sequestering ssDNA, detecting DNA damage, stimulating strand exchange proteins, nucleases and helicases, activating transcription and mediation of protein-protein interactions. The SSB family of proteins are structurally and functionally highly conserved through evolution. In bacteria and archaea they are involved in a host of processes including DNA damage repair, DNA replication and transcription. The major SSB homologue in eukaryotes, namely the Replication Protein A (RPA), is a heterotrimer and is required for both DNA replication and repair.

Prior to the work leading to the present invention, RPA was considered to be the sole or primary eukaryotic SSB. The present applicant has, however, identified and described hereinafter, novel human SSBs, designated hSSB1 and hSSB2. These proteins have a domain organisation that is closer to the archaeal SSB than to eukaryotic RPA, but hSSB1 at least, behaves in a manner that is characteristic of so-called DNA double strand break (DSB) sensors (Zhou and Elledge, 2000). As shown in the Examples, upon induction of DNA damage, hSSB1 accumulates in the nucleus, forming distinct foci that co-localise with known repair proteins. It has also been observed that depletion of hSSB1 abrogates the cellular response to DSBs, including activation of the ATM protein kinase (ATM) and phosphorylation of ATM targets after exposure to ionising radiation (IR). Further, it has been found that hSSB1 is associated with the Mre11-Rad50-Nbs1 (MRN) complex and that hSSB1-deficient cells are defective in the recruitment of the MRN complex to sites of DNA breaks. More particularly, it has been found that hSSB1 interacts with the MRN complex and facilitates the recruitment of this complex, and other factors, to foci at the site of DNA damage. Further, it has been found that hSSB1 is involved in generating and maintaining stability in ssDNA formed after DNA damage and, thus, appears to contribute to repair by homologous recombination (HR). Moreover, cells deficient in hSSB1 exhibit increased radiosensitivity and enhanced genomic instability coupled with a diminished capacity for DNA repair, thereby indicating that a loss of hSSB1 impairs DNA damage response.

As an early participant in the damage response pathway, hSSB1 is accordingly implicated in tumourigenesis, thus providing a suitable marker for cancer diagnosis, cancer predisposition and the prognosis of existing cancers or tumours. Further, it is considered that “hSSB1 status” (e.g. detection of hSSB1 overexpression) can provide an indication of potential tumour response to various cancer treatments.

### SUMMARY OF THE INVENTION

In a first aspect, the present invention provides a method of detecting transformed cells or tumour cells comprising the step of detecting in a suitable biological sample, overexpression of a human ssDNA binding (SSB) protein or polypeptide comprising the following amino acid sequence:

(SEQ ID NO: 1)

FX¹X²DX³KPGLKNLNX⁴X⁵FIVLEX⁶GRVTKTKDGHEVRX⁷CKVADKT
GSIX⁸ISVWDX⁹X¹⁰GX¹¹LIQPGDIIRLTX¹²GYASX¹³X¹⁴KGCLTL
YTGRGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof.

The method of the first aspect may be used, for example, for diagnosing or prognosing cancer or assessing a predisposition to cancer. The method may also be used in selecting a suitable cancer treatment or in assessing the effectiveness of a cancer treatment.

In a second aspect, the present invention provides a method of diagnosing or prognosing cancer or assessing a predisposition to cancer, said method comprising the step of detecting in a suitable biological sample from a subject, overexpression of a human ssDNA binding (SSB) protein or polypeptide comprising the following amino acid sequence:

(SEQ ID NO: 1)

FX¹X²DX³KPGLKNLNX⁴X⁵FIVLEX⁶GRVTKTKDGHEVRX⁷CKVADKT
GSIX⁸ISVWDX⁹X¹⁰GX¹¹LIQPGDIIRLTX¹²GYASX¹³X¹⁴KGCLTL
YTGRGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof.

The method of the second aspect is preferably used for diagnosing or prognosing breast or bowel cancer or assessing a predisposition to breast or bowel cancer.

In the methods of the invention, the said SSB protein or polypeptide is preferably a human SSB1 protein or polypeptide comprising an amino acid sequence substantially corresponding to the following:

(SEQ ID NO: 2)

MTTETFVKDIKPGLKNLNLIFIVLETGRVTKTKDGHEVRTCKVADKTG
SINISVWDDVGNLIQPGDIIRLTKGYASVFKGCLTLYTGRGGDLQKIGEF
CMVYSEVPNFSEPNPEYSTQQAPNKAVQNDSNPSASQPTTGPSAASPA
SENQNGNGLSAPPGPGGGPHPPHTPSHPPSTRITRSQPNHTPAGPPGPS
SNPVSNGKETRRSSKR,

or a naturally occurring variant sequence thereof.

Also, in the methods of the invention, the step of detecting overexpression of said SSB protein or polypeptide may comprise indirectly detecting overexpression of the protein or polypeptide by determining the relative amount of messenger RNA (mRNA) encoding the protein or polypeptide that is present in said sample. However, more preferably, the step of detecting overexpression of said SSB protein or polypeptide comprises directly detecting overexpression of the protein or polypeptide by determining the relative amount of the protein or polypeptide per se (or a fragment thereof) that is present in the said sample.

For directly detecting overexpression of the SSB protein or polypeptide, preferably an antibody or fragment thereof that is capable of specifically binding with the protein or polypeptide (or a fragment thereof), is used in determining the relative amount of the protein or polypeptide that is present in the sample (e.g. by using standard ELISA methods).

Thus, in a third aspect, the present invention provides an antibody or fragment thereof which specifically binds to a human ssDNA binding (SSB) protein or polypeptide comprising the following amino acid sequence:

(SEQ ID NO: 1)

FX¹X²DX³KPGLKNLNX⁴X⁵FIVLEX⁶GRVTKTKDGHEVRX⁷CKVADK
TGSIX⁸ISVWDX⁹X¹⁰GX¹¹LIQPGDIIRLTX¹²GYASX¹³X¹⁴KGCLTL
YTGRGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof;
or said antibody or fragment thereof binds to an antigenic fragment of said protein or polypeptide.

In a fourth aspect, the present invention provides an isolated human ssDNA binding (SSB) protein or polypeptide comprising the following amino acid sequence:

(SEQ ID NO: 1)

FX¹X²DX³KPGLKNLNX⁴X⁵FIVLEX⁶GRVTKTKDGHEVRX⁷CKVADKTG
SIX⁸ISVWDX⁹X¹⁰GX¹¹LIQPGDIIRLTX¹²GYASX¹³X¹⁴KGCLTLY
TGRGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof;
or an antigenic fragment thereof.

In a fifth aspect, the present invention provides an isolated polynucleotide molecule encoding a human ssDNA binding (SSB) protein or polypeptide comprising the following amino acid sequence:

(SEQ ID NO: 1)

FX¹X²DX³KPGLKNLNX⁴X⁵FIVLEX⁶GRVTKTKDGHEVRX⁷CKVADKTG
SIX⁸ISVWDX⁹X¹⁰GX¹¹LIQPGDIIRLTX¹²GYASX¹³X¹⁴KGCLTLYT
GRGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof.

In a sixth aspect, the present invention provides an oligonucleotide molecule suitable for use as, for example, a probe or primer sequence which hybridises under high stringency conditions to a polynucleotide molecule encoding a human ssDNA binding (SSB) protein or polypeptide comprising the following amino acid sequence:

(SEQ ID NO: 1)

FX¹X²DX³KPGLKNLNX⁴X⁵FIVLEX⁶GRVTKTKDGHEVRX⁷CKVADKT
GSIX⁸ISVWDX⁹X¹⁰GX¹¹LIQPGDIIRLTX¹²GYASX¹³X¹⁴KGCLTL
YTGRGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof.

In a seventh aspect, the present invention provides a kit for diagnosing or prognosing cancer or assessing a predisposition to cancer, wherein said kit comprises any one or a combination of:

(i) an isolated eukaryotic SSB protein or polypeptide,
(ii) an antibody or fragment thereof according to the third aspect, and
(iii) an oligonucleotide molecule suitable for use as a probe or primer sequence, according to the sixth aspect.

Homologues of the sequence shown above as SEQ ID NO: 2 have been identified in other divergent eukaryotic species (see FIG. 1).

Thus, in a further aspect, the present invention provides an isolated eukaryotic ssDNA binding (SSB) protein or polypeptide comprising the following amino acid sequence:

(SEQ ID NO: 3)

X^{A}X¹X²DX³KX^{B}GX^{C}KNX^{D}X^{E}X⁴X⁵FIVLEX⁶GX^{F}X^{G}TX^{H}TKX^{I}X^{J}X^{K}
EVRX⁷X^{L}X^{M}VX^{N}DX^{O}X^{P}X^{Q}X^{R}IX⁸X^{S}SX^{T}WDX⁹X¹⁰GX¹¹X^{U}IX^{V}X^{W}GDI
X^{X}RLTX¹²GYASX¹³X¹⁴X^{Y}X^{Z}CLTLYX^{AB}GX^{AC}X^{AD}GX¹⁵X^{AE}X^{AF}KIG
EX^{AG}CMVX^{AH}X^{AI}EX^{AJ}X^{AK}NX^{AL}SEPX^{AM}X^{AN}X¹⁶X^{AO}X¹⁷X¹⁸QX^{AP}

wherein
X^{A} is selected from F, L and P, X¹ is selected from V and I, X² is selected from K and R, X³ is selected from I and V, X^{B} is selected from P and A, X^{C} is selected from L and S, X^{D} is selected from L and I, X^{E} is selected from N and S, X⁴ is selected from L, V and I, X⁵ is selected from I, L and V, X⁶ is selected from T, I and V, X^{F} is selected from R and V, X^{G} is selected from V and A, X^{H} is selected from K and V, X^{I} is selected from D and E, X^{J} is selected from G and N, X^{K} is selected from H and R, X⁷ is selected from T, S and N, X^{L} is selected from C and F, X^{M} is selected from K and R, X^{N} is selected from A and G, X^{O} is selected from K, R and P, X^{P} is selected from T and S, X^{Q} is selected from G and A, X^{R} is selected from S and C, X⁸ is selected from N, T and A, X^{S} is selected from I and V, X^{T} is selected from V and I, X⁹ is selected from D and E, X¹⁰ is selected from V, I, L and P, X¹¹ is selected from N, G, S and K, X^{U} is selected from L and F, X^{V} is selected from Q and A, X^{W} is selected from P and T, X^{X} is selected from I and V, X¹² is selected from K and R, X¹³ is selected from V, M, L and I, X¹⁴ is selected from F and W, X^{Y} is selected from K and R, X^{Z} is selected from G and H, X^{AB} is selected from T and S, X^{AC} is selected from R and K, X^{AD} is selected from G and N, X¹⁵ is selected from D and E, X^{AE} is selected from L and V, X^{AF} is selected from Q and F, X^{AG} is selected from F and Y, X^{AH} is selected from Y and F, X^{AI} is selected from S and N, X^{AJ} is selected from V and S, X^{AK} is selected from P and V, X^{AL} is selected from F and M, X^{AM} is selected from N and K, X^{AN} is P or is null, X¹⁶ is selected from E and D or is null, X^{AO} is selected from Y, L and R, X¹⁷ is selected from S, R, N, I, L and A, X¹⁸ is selected from T, G, A and E, and X^{AP} is selected from Q and A, or a naturally occurring variant sequence thereof;
or an antigenic fragment thereof.

In a still further aspect, the present invention provides a polynucleotide molecule or oligonucleotide molecule comprising a nucleotide sequence encoding all or part of a eukaryotic SSB protein or polypeptide comprising an amino acid sequence as shown above as SEQ ID NO: 3, and/or the complementary sequence thereto.

### BRIEF DESCRIPTION OF THE FIGURES

FIG. 1 (A) shows the nucleotide and amino acid sequence for the hSSB1 protein, and (B) shows the nucleotide and amino acid sequence for the hSSB2 protein, as retrieved using the BLAST algorithm from the NCBI database, while (C) shows an alignment of the hSSB1 and hSSB2 amino acid sequences (designated in the figure as “human 1” and “human 2” respectively) against that of archaeal SSB (Sulfolobus solfataricus), the corresponding “mouse 1” and “mouse 2” amino acid sequences, as well as the amino acid sequences of the homologues from Xenopus laevis, Danio rerio and Drosophila melanogaster. The alignment indicates that the proteins have a highly conserved N-terminal domain (an oligonucleotide/oligosaccharide-binding (OB-fold) domain) followed by a variable region with no predicted structure and a conserved C-terminal tail.

FIG. 2 shows the binding of recombinant hSSB1 to ssDNA substrate (top) and a synthetic replication fork (bottom) by electrophoretic mobility shift assay (EMSA). The location of the radiolabel is marked with a filled circle.

FIG. 3 shows Western immunoblot analysis of hSSB1 and actin (control) using cell extracts from neonatal foreskin fibroblast (NFF) cells exposed to IR (6 Gy) or UV (20 mJ/m2) light at 0, 0.5, 1, 1.5, 2 and 3 hours time points.

FIG. 4 shows metaphase control in hSSB1-deficient and control NFF cells; chromosome breaks are indicated by arrows.

FIG. 5 shows the frequency of spontaneous and IR (2 Gy) induced chromosomal aberrations in control and hSSB1-deficient NFF cells. Dose of IR is represented on the X axis and the relative number of aberrations at metaphase is represented on the Y axis.

FIG. 6 shows control and hSSB1-deficient NFF cells at the G₁/S checkpoint following IR exposure. From left, panels show cells transfected with control siRNA, cells transfected with control siRNA and exposed to 6 Gy IR, cells transfected with hSSB1-specific siRNA and cells transfected with hSSB1-specific siRNA and exposed to 6 Gy IR. The boxed area shows bromodeoxyuridine (BrdUrd) positive cells.

FIG. 7 shows IR sensitivity in control and hSSB1-depleted NFF cells. Dose of IR is represented on the X axis and relative cell survival is represented on the Y axis.

FIG. 8 shows the localisation of hSSB1 to DNA repair foci after IR (6 Gy).

FIG. 9 shows hSSB1 formation of foci that co-localise with γH2AX (top panel). hSSB1 and γH2AX co-localise at a single double strand break (DSB) induced by the I-SceI restriction enzyme in MCF7 DRGFP cells (bottom panel).

FIG. 10 shows the co-localisation of hSSB1 with foci formed by Rad50 and Mre11.

FIG. 11 shows NBS1 and Rad50 foci formation in control and hSSB1-depleted NFF cells.

FIG. 12 shows Rad51 foci formation in control and hSSB1-depleted NFF cells.

FIG. 13 shows H2AX foci formation in control and hSSB1-depleted NFF cells.

FIG. 14 shows IR induced activation of ATM and the subsequent phosphorylation of downstream targets Nbs 1, p53, Chk1 and Chk2 in control and hSSB1-depleted NFF cells.

FIG. 15 shows IR induced phosphorylation of γH2AX in control and hSSB1-depleted NFF cells.

FIG. 16 shows ChIP analysis of hSSB1 enrichment on a unique DSB induced by I-SceI in vivo. The Y axis scale represents protein enrichment relative to baseline measures.

FIG. 17 shows IR induced ssDNA foci formation in control and hSSB1-specific siRNA transfected cells.

FIG. 18 shows HR repair events in cells transfected with hSSB1 siRNA in response to an I-SceI-induced DSB as determined by FACS analysis. The Y axis scale represents the relative number of I-Sce1 induced homologous recombination repair (HRR) events.

FIG. 19 shows the survival rate of patients expressing hSSB1 in comparison to patients not expressing hSSB1 (hSSB1 positive shown as “1SSB pos”, and hSSB1 negative shown as “1SSB neg”).

### DETAILED DESCRIPTION OF THE INVENTION

The present applicant has found that hSSB1 is involved in generating and maintaining genomic stability and signal transduction following DNA damage and thus contributes to DNA repair. Further, cells deficient in hSSB1 exhibit a diminished capacity for DNA repair, indicating that a loss of hSSB1 impairs DNA damage responses. As an early participant in the damage response pathway, hSSB1 is accordingly implicated in cellular transformation and tumorigenesis thus providing a suitable marker for cancer diagnosis, cancer predisposition and the prognosis of existing cancers or tumours. Further, hSSB1 status can provide an indication of potential tumour response to various cancer treatments thus finding application in the selection of suitable treatments or treatment regimes. In a similar manner, hSSB1 status may be used to assess the effectiveness of a cancer treatment. It is anticipated that the closely related hSSB2 protein provides a marker with similar utilities.

Thus, in a first aspect, the present invention provides a method of detecting transformed cells or tumour cells comprising the step of detecting in a suitable biological sample, overexpression of a human ssDNA binding (SSB) protein or polypeptide comprising the following amino acid sequence:

(SEQ ID NO: 1)

FX¹X²DX³KPGLKNLNX⁴X⁵FIVLEX⁶GRVTKTKDGHEVRX⁷CKVADKTG
SIX⁸ISVWDX⁹X¹⁰GX¹¹LIQPGDIIRLTX¹²GYASX¹³X¹⁴KGCLTLYT
GRGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof.

As mentioned above, the method of the first aspect may be used in selecting a suitable cancer treatment or in assessing the effectiveness of a cancer treatment. In particular, the detection of transformed cells or tumour cells through the detection of overexpression of a human ssDNA binding (SSB) protein or polypeptide in a suitable biological sample, can be used to assist selection of a suitable cancer treatment by omitting from the group of possible treatments those involving radiotherapy and/or DNA damaging chemotherapies.

In a second aspect, the present invention provides a method of diagnosing or prognosing cancer or assessing a predisposition to cancer, said method comprising the step of detecting in a suitable biological sample from a subject, overexpression of a human ssDNA binding (SSB) protein or polypeptide comprising the following amino acid sequence:

(SEQ ID NO: 1)

FX¹X²DX³KPGLKNLNX⁴X⁵FIVLEX⁶GRVTKTKDGHEVRX⁷CKVADKTG
SIX⁸ISVWDX⁹X¹⁰GX¹¹LIQPGDIIRLTX¹²GYASX¹³X¹⁴KGCLTLYT
GRGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof.

The detection of overexpression of the SSB protein or polypeptide in the suitable biological sample can be used, in the case of a subject in which cancer has not previously been diagnosed, either on its own or in combination with other cancer tests, to diagnose cancer in the subject. For a subject having already been diagnosed as having cancer, the detection of overexpression of the SSB protein or polypeptide in the suitable biological sample can be indicative of the prognosis of that cancer (i.e. the greater the relative level of SSB expression, the worse the prognosis of the cancer). Further, for a subject in which cancer has not previously been diagnosed and who is not showing any signs of ill health due to cancer, the detection of overexpression of the SSB protein or polypeptide in the suitable biological sample can be used in an assessment of a predisposition to cancer (i.e. SSB overexpression is likely to indicate that the subject is predisposed to the development of cancer).

For prognosing cancer, the method of the second aspect may further comprise determining the intracellular location(s) of the SSB protein or polypeptide in a transformed cell or tumour cell in the suitable biological sample. That is, a determination that the SSB protein or polypeptide is present in the cytoplasm of such cells, and not merely the nucleus, can be used to provide a worse prognosis of the cancer.

The method of the second aspect is preferably used for diagnosing or prognosing breast or bowel cancer or assessing a predisposition to breast or bowel cancer.

In the methods of the invention, the said SSB protein or polypeptide is preferably a human SSB1 protein or polypeptide comprising an amino acid sequence substantially corresponding to the following:

(SEQ ID NO: 2)

MTTETFVKDIKPGLKNLNLIFIVLETGRVTKTKDGHEVRTCKVADKTGSI
NISVWDDVGNLIQPGDIIRLTKGYASVFKGCLTLYTGRGGDLQKIGEFC
MVYSEVPNFSEPNPEYSTQQAPNKAVQNDSNPSASQPTTGPSAASPASEN
QNGNGLSAPPGPGGGPHPPHTPSHPPSTRITRSQPNHTPAGPPGPSSNP
VSNGKETRRSSKR;

or a naturally occurring variant sequence thereof.

However, alternatively, the said SSB protein or polypeptide is a human SSB2 protein or polypeptide comprising an amino acid sequence substantially corresponding to the following:

(SEQ ID NO: 4)

MNRVNDPLIFIRDIKPGLKNLNVVFIVLEIGRVTKTKDGHEVRSCKVADK
TGSITISVWDEIGGLIQPGDIIRLTRGYASMWKGCLTLYTGRGGELQKIG
EFCMVYSEVPNFSEPNPDYRGQQNKGAQSEQKNNSMNSNMGTGTFGPV
GNGVHTGPESREHQFSHAGRSNGRGLINPQLQGTASNQTV;

or a naturally occurring variant sequence thereof.

As used herein, the term “naturally occurring variant sequence” refers to the sequence of any naturally occurring isoform of the relevant SSB protein or polypeptide, encoded by, for example, an allelic variant. The variant sequence may, therefore, encompass one or more amino acid substitutions, deletions and/or additions, but would generally vary from the relevant amino acid sequence by no more than five amino acids.

The term “substantially corresponding” as used herein in relation to amino acid sequences is to be understood as encompassing minor variations in the relevant amino acid sequence which do not result in any significant alteration of the biological activity of the SSB protein or polypeptide. These variations may include conservative amino acid substitutions such as: G, A, V, I, L, M; D, E; N, Q:S, T:K, R, H; F, Y, W, H; and P, Nα-alkylamino acids.

The step of detecting overexpression of said SSB protein or polypeptide may comprise indirectly detecting overexpression of the protein or polypeptide by determining the relative amount of messenger RNA (mRNA) encoding the protein or polypeptide that is present in said sample. The relative amount of mRNA encoding the protein or polypeptide may be determined by any of the methods well known to persons skilled in the art including Northern blot (by comparison to reference samples) and PCR-based mRNA quantification methods (e.g. using RT-PCR with primers conjugated to a detectable label). Generally, the relative amount of mRNA encoding the protein or polypeptide will be determined by comparison against the amount, or range of amounts, present in “normal samples” (e.g. samples from the subject that do not include transformed cells or tumour cells, or otherwise, equivalent biological samples taken from normal subject(s)). The step of detecting overexpression of said SSB protein or polypeptide may also comprise indirectly detecting overexpression of the protein or polypeptide by determining the relative amount of an antibody or fragment thereof that specifically binds to the SSB protein or polypeptide. The relative amount of such an antibody or fragment thereof may be determined by any of the methods well known to persons skilled in the art including (e.g. standard ELISA methods). As such, the relative amount of an antibody or fragment thereof that specifically binds to the SSB protein or polypeptide can be determined by quantitatively detecting the antibody or fragment thereof with, for example, SSB protein or polypeptide which may be immobilised or conjugated to a detectable label. Suitable detectable labels include chromophores, fluorophores (e.g. fluorescein or FITC), radiolabels (e.g. ¹²⁵I), and enzymes such as horseradish peroxidase. Generally, the relative amount of the antibody or fragment thereof will be determined by comparison against the amount, or range of amounts, present in “normal samples” (e.g. equivalent biological samples taken from normal subject(s)).

Preferably, the step of detecting overexpression of said SSB protein or polypeptide comprises directly detecting overexpression of the protein or polypeptide by determining the relative amount of the protein or polypeptide per se (or a fragment thereof) that is present in the said sample. For directly detecting overexpression of the SSB protein or polypeptide, preferably an antibody or fragment thereof that is capable of specifically binding with the protein or polypeptide (or a fragment thereof), is used in determining the relative amount of the protein or polypeptide that is present in the sample. This can be achieved by using any of the methods well known to persons skilled in the art (e.g. standard ELISA methods or in situ immunofluorescence using tissue section samples). As such, the relative amount of the SSB protein or polypeptide can be determined by quantitatively detecting the protein or polypeptide with a specific antibody or fragment thereof (i.e. a primary antibody) which is either directly conjugated to a detectable label or is otherwise detected via a secondary antibody or fragment thereof directly conjugated to a detectable label. Suitable detectable labels include those mentioned above. These labels can be used in methods and systems as are well known to persons skilled in the art, which provide for the automation or partial automation of the step of detecting overexpression of the SSB protein or polypeptide (e.g. by a microplate reader or use of a flow cytometer).

The suitable biological sample may be selected from, for example, tissue biopsies and fixed sections (e.g. formalin fixed or paraffin embedded) or fixed cell samples prepared therefrom, smear samples, blood samples, faecal samples, urine samples or buccal samples. The sample may be pre-treated by, for example, filtration, separation or extraction methods to partly or completely purify or isolate cells, proteins, polynucleotides, oligonucleotides or fragments thereof or fractions containing these components.

In a third aspect, the present invention provides an antibody or fragment thereof which specifically binds to a human ssDNA binding (SSB) protein or polypeptide comprising the following amino acid sequence:

(SEQ ID NO: 1)

FX¹X²DX³KPGLKNLNX⁴X⁵FIVLEX⁶GRVTKTKDGHEVRX⁷CKVADKTG
SIX⁸ISVWDX⁹X¹⁰GX¹¹LIQPGDIIRLTX¹²GYASX¹³X¹⁴KGCLTLYT
GRGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof;
or said antibody or fragment thereof binds to an antigenic fragment of said protein or polypeptide.

The antibody may be selected from monoclonal and polyclonal antibodies.

The antibody fragment may be selected from fragments produced through enzymatic cleavage such as Fab and F(ab′)₂ fragments, and recombinant antibody fragments such as single chain Fv (scFv) fragments.

In a fourth aspect, the present invention provides an isolated human ssDNA binding (SSB) protein or polypeptide comprising the following amino acid sequence:

(SEQ ID NO: 1)

X¹X²DX³KPGLKNLNX⁴X⁵FIVLEX⁶GRVTKTKDGHEVRX⁷CKVADKTGS
IX⁸ISVWDX⁹X¹⁰GX¹¹LIQPGDIIRLTX¹²GYASX¹³X¹⁴KGCLTLYTG
RGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof;
or an antigenic fragment thereof.

The protein, polypeptide or antigenic fragment of the invention may be isolated from a suitable biological sample from a subject, or may otherwise be prepared recombinantly and thereafter isolated from a recombinant cell culture.

The protein, polypeptide or antigenic fragment may be used, for example, to immunise a suitable animal (e.g. mouse, rabbit or sheep) in order produce an antibody or fragment thereof according to the third aspect. To this end, the protein, polypeptide or antigenic fragment may optionally be fused to a suitable carrier protein such as human serum albumin to form an immunogen.

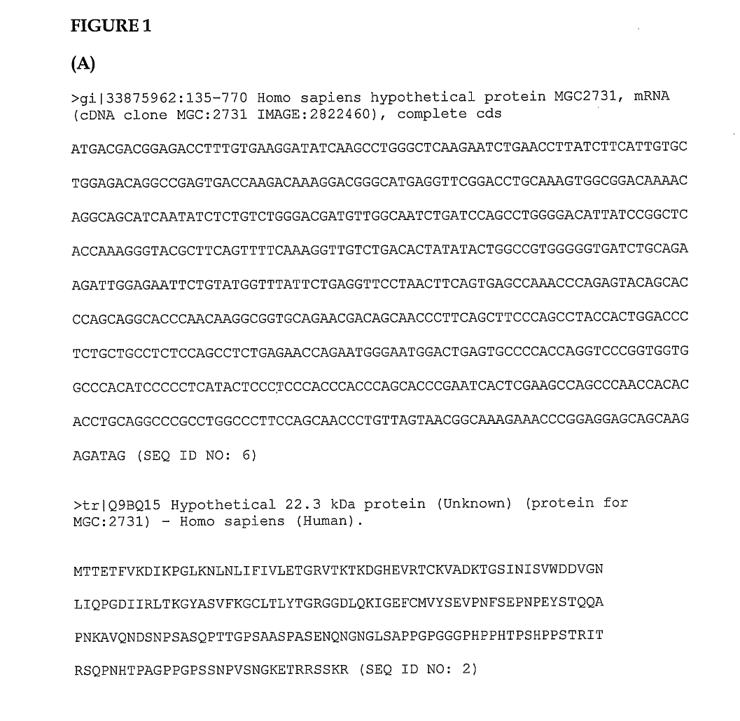

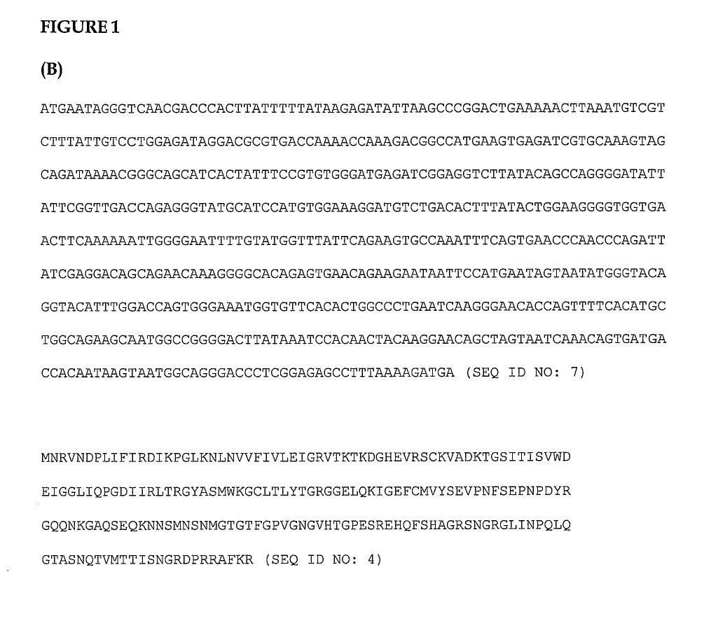

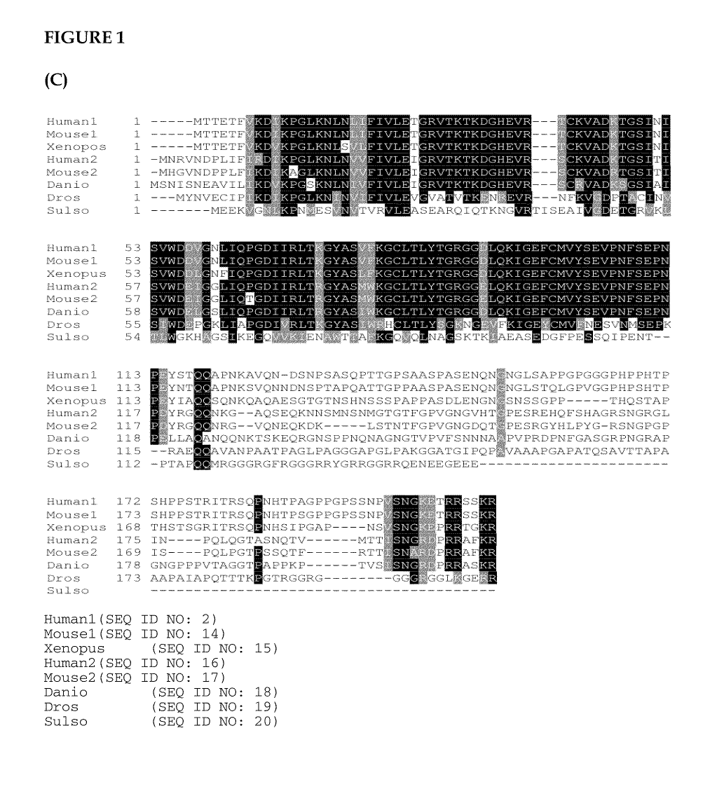

Suitable antigenic fragments will typically comprise an amino acid sequence derived from a non-conserved C-terminal region of the SSB protein or polypeptide (see FIG. 1). A particular example of a suitable antigenic fragment to produce an antibody specific for the hSSB1 protein or polypeptide comprises the following amino acid sequence:

| | |
|---|---|
| NPEYSTQQAPN | (SEQ ID NO: 5) |

In a fifth aspect, the present invention provides an isolated polynucleotide molecule encoding a human ssDNA binding (SSB) protein or polypeptide comprising the following amino acid sequence:

(SEQ ID NO: 1)

FX¹X²DX³KPGLKNLNX⁴X⁵FIVLEX⁶GRVTKTKDGHEVRX⁷CKVADKTG
SIX⁸ISVWDX⁹X¹⁰GX¹¹LIQPGDIIRLTX¹²GYASX¹³X¹⁴KGCLTLYT
GRGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof.

Preferably, the polynucleotide molecule comprises a nucleotide sequence encoding a human SSB protein or polypeptide comprising an amino acid sequence substantially corresponding to the sequence shown above as SEQ ID NO: 2 or a naturally occurring variant sequence thereof, or that shown above as SEQ ID NO: 4 or a naturally occurring variant sequence thereof.

More preferably, the polynucleotide molecule encodes an hSSB1 protein or polypeptide and comprises a nucleotide sequence substantially corresponding to the following:

(SEQ ID NO: 6)
ATGACGACGGAGACCTTTGTGAAGGATATCAAGCCTGGGCTCAAGAATCTGAACCTTATCTTCATTG
TGCTGGAGACAGGCCGAGTGACCAAGACAAAGGACGGGCATGAGGTTCGGACCTGCAAAGTGGCGGA
CAAAACAGGCAGCATCAATATCTCTGTCTGGGACGATGTTGGCAATCTGATCCAGCCTGGGGACATT
ATCCGGCTCACCAAAGGGTACGCTTCAGTTTTCAAAGGTTGTCTGACACTATATACTGGCCGTGGGG
GTGATCTGCAGAAGATTGGAGAATTCTGTATGGTTTATTCTGAGGTTCCTAACTTCAGTGAGCCAAA
CCCAGAGTACAGCACCCAGCAGGCACCCAACAAGGCGGTGCAGAACGACAGCAACCCTTCAGCTTCC
CAGCCTACCACTGGACCCTCTGCTGCCTCTCCAGCCTCTGAGAACCAGAATGGGAATGGACTGAGTG
CCCCACCAGGTCCCGGTGGTGGCCCACATCCCCCTCATACTCCCTCCCACCCACCCAGCACCCGAAT
CACTCGAAGCCAGCCCAACCACACACCTGCAGGCCCGCCTGGCCCTTCCAGCAACCCTGTTAGTAAC
GGCAAAGAAACCCGGAGGAGCAGCAAGAGATAG,

and/or the complementary sequence thereto.

Alternatively, the polynucleotide molecule encodes an hSSB2 protein or polypeptide and comprises a nucleotide sequence substantially corresponding to the following:

(SEQ ID NO: 7)
ATGAATAGGGTCAACGACCCACTTATTTTTATAAGAGATATTAAGCCCGGACTGAAAAACTTAAATG
TCGTCTTTATTGTCCTGGAGATAGGACGCGTGACCAAAACCAAAGACGGCCATGAAGTGAGATCGTG
CAAAGTAGCAGATAAAACGGGCAGCATCACTATTTCCGTGTGGGATGAGATCGGAGGTCTTATACAG
CCAGGGGATATTATTCGGTTGACCAGAGGGTATGCATCCATGTGGAAAGGATGTCTGACACTTTATA
CTGGAAGGGGTGGTGAACTTCAAAAAATTGGGGAATTTTGTATGGTTTATTCAGAAGTGCCAAATTT
CAGTGAACCCAACCCAGATTATCGAGGACAGCAGAACAAAGGGGCACAGAGTGAACAGAAGAATAAT
TCCATGAATAGTAATATGGGTACAGGTACATTTGGACCAGTGGGAAATGGTGTTCACACTGGCCCTG
AATCAAGGGAACACCAGTTTTCACATGCTGGCAGAAGCAATGGCCGGGGACTTATAAATCCACAACT
ACAAGGAACAGCTAGTAATCAAACAGTGATGACCACAATAAGTAATGGCAGGGACCCTCGGAGAGCC
TTTAAAAGATGA,

and/or the complementary sequence thereto.

The term “substantially corresponding” as used herein in relation to nucleotide sequences is to be understood as encompassing minor variations in the relevant nucleotide sequence which, due to degeneracy in the DNA code, do not result in a change in the encoded SSB protein or polypeptide. Further, the term is to be understood as encompassing minor variations in the relevant nucleotide sequence which may be required in order to enhance expression in a particular system (i.e. to comply with preferred codon usage) but which do not otherwise result in any significant alteration of the biological activity of the SSB protein or polypeptide.

In a sixth aspect, the present invention provides an oligonucleotide molecule which hybridises under high stringency conditions to a polynucleotide molecule encoding a human ssDNA binding (SSB) protein or polypeptide comprising the following amino acid sequence:

(SEQ ID NO: 1)

FX¹X²DX³KPGLKNLNX⁴X⁵FIVLEX⁶GRVTKTKDGHEVRX⁷CKVADKTG
SIX⁸ISVWDX⁹X¹⁰GX¹¹LIQPGDIIRLTX¹²GYASX¹³X¹⁴KGCLTLYT
GRGGX¹⁵LQKIGEFCMVYSEVPNFSEPNPX¹⁶YX¹⁷X¹⁸QQ

wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof.

“High stringency conditions” are well known to persons skilled in the art, and are typically characterised by high temperature (i.e. high annealing temperature) and low ionic strength (i.e. low salt concentration, especially of MgCl₂, KCl and NaCl). The high stringency conditions may vary according to the circumstances of the hybridisation (i.e. for probe hybridisation, PCR amplification, etc.). For the purposes of the present invention, as defined by the sixth aspect, “high stringency conditions” is to be understood as referring to such conditions applicable to probe hybridisation (e.g. conditions which: (1) employ low ionic strength and high temperature for washing, for example, 15 mM NaCl/1.5 mM sodium citrate/0.1% NaDodSO₄ at 50° C.; (2) employ, during hybridisation, a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% form amide, 5×SSC (750 mM NaCl, 75 mM sodium citrate), 50 mM sodium phosphate (pH 6.8), 0.1% sodium pyrophosphate, 5×Denhardt's solution, sonicated salmon sperm DNA (50 μg/ml), 0.1% SDS and 10% dextran sulfate at 42° C. in 0.2×SSC (30 mM NaCl, 3 mM sodium citrate) and 0.1% SDS).

Such an oligonucleotide molecule may be suitable for use as, for example, a probe or primer sequence, or may consist as an antisense oligonucleotide molecule (e.g. antisense RNA or DNA, which may include catalytic sequences such as those well known to persons skilled in the art, or a small interfering RNA (siRNA) molecule).

The oligonucleotide molecule will typically consist of 10 to 50 nucleotides and, more preferably, about 15 to 30 nucleotides.

Preferably, the oligonucleotide molecule is derived from the nucleotide sequence shown above as SEQ ID NO: 2 or a naturally occurring variant sequence thereof (or the complementary sequence thereto), or that shown above as SEQ ID NO: 4 or a naturally occurring variant sequence thereof (or the complementary sequence thereto).

One particular example of an oligonucleotide molecule of the present invention comprises the following nucleotide sequence:

| | |
|---|---|
| GACAAAGGACGGGCATGAGTT. | (SEQ ID No: 8) |

Another particular example of an oligonucleotide molecule of the present invention comprises a siRNA molecule according to the following structure:

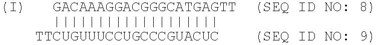

The isolated polynucleotide or oligonucleotide molecule of the invention may be provided in the form of an isolated expression vector or expression cassette comprising an operably linked promoter sequence oriented to produce sense transcripts (e.g. for expression of an SSB protein or polypeptide) or antisense transcripts (e.g. to produce antisense RNA). For the production of siRNA, a suitable oligonucleotide molecule may be operably linked with, for example, a U6 or H1 RNA polymerase III promoter sequence as is well known to persons skilled in the art.

In a seventh aspect, the present invention provides a kit for diagnosing or prognosing cancer or assessing a predisposition to cancer, wherein said kit comprises any one or a combination of:

(i) an isolated eukaryotic SSB protein or polypeptide,
(ii) an antibody or fragment thereof according to the third aspect, and
(iii) an oligonucleotide molecule suitable for use as a probe or primer sequence, according to the sixth aspect.

Preferably, the kit comprises a primary antibody which specifically binds with a human SSB protein or polypeptide (especially an hSSB1 protein or polypeptide) and a secondary antibody conjugated to a detectable label which binds to said primary anybody.

The kit may further comprise various buffer solutions as will be apparent to persons skilled in the art.

As mentioned above, homologues of the sequence shown above as SEQ ID NO: 2 have been identified in other divergent eukaryotic species.

Thus, in a further aspect, the present invention provides an isolated eukaryotic ssDNA binding (SSB) protein or polypeptide comprising the following amino acid sequence:

(SEQ ID NO: 3)

X^{A}X¹X²DX³KX^{B}GX^{C}KNX^{D}X^{E}X⁴X⁵FIVLEX⁶GX^{F}X^{G}TX^{H}TKX^{I}X^{J}X^{K}EV
RX⁷X^{L}X^{M}VX^{N}DX^{O}X^{P}X^{Q}X^{R}IX⁸X^{S}SX^{T}WDX⁹X¹⁰GX¹¹X^{U}IX^{V}X^{W}GDIX^{X}
RLTX¹²GYASX¹³X¹⁴X^{Y}X^{Z}CLTLYX^{AB}GX^{AC}X^{AD}GX¹⁵X^{AE}X^{AF}KIG
EX^{AG}CMVX^{AH}X^{AI}EX^{AJ}X^{AK}NX^{AL}SEPX^{AM}X^{AN}X¹⁶X^{AO}X¹⁷X¹⁸QX^{AP}

wherein
X^{A} is selected from F, L and P, X¹ is selected from V and I, X² is selected from K and R, X³ is selected from I and V, X^{B} is selected from P and A, X^{C} is selected from L and S, X^{D} is selected from L and I, X^{E} is selected from N and S, X⁴ is selected from L, V and I, X⁵ is selected from I, L and V, X⁶ is selected from T, I and V, X^{F} is selected from R and V, X^{G} is selected from V and A, X^{H} is selected from K and V, X^{I} is selected from D and E, X^{J} is selected from G and N, X^{K} is selected from H and R, X⁷ is selected from T, S and N, X^{L} is selected from C and F, X^{M} is selected from K and R, X^{N} is selected from A and G, X^{O} is selected from K, R and P, X^{P} is selected from T and S, X^{Q} is selected from G and A, X^{R} is selected from S and C, X⁸ is selected from N, T and A, X^{S} is selected from I and V, X^{T} is selected from V and I, X⁹ is selected from D and E, X¹⁰ is selected from V, I, L and P, X¹¹ is selected from N, G, S and K, X^{U} is selected from L and F, X^{V} is selected from Q and A, X^{W} is selected from P and T, X^{X} is selected from I and V, X¹² is selected from K and R, X¹³ is selected from V, M, L and I, X¹⁴ is selected from F and W, X^{Y} is selected from K and R, X^{Z} is selected from G and H, X^{AB} is selected from T and S, X^{AC} is selected from R and K, X^{AD} is selected from G and N, X¹⁵ is selected from D and E, X^{AE} is selected from L and V, X^{AF} is selected from Q and F, X^{AG} is selected from F and Y, X^{AH} is selected from Y and F, X^{AI} is selected from S and N, X^{AJ} is selected from V and S, X^{AK} is selected from P and V, X^{AL} is selected from F and M, X^{AM} is selected from N and K, X^{AN} is P or is null, X¹⁶ is selected from E and D or is null, X^{AO} is selected from Y, L and R, X¹⁷ is selected from S, R, N, I, L and A, X¹⁸ is selected from T, G, A and E, and X^{AP} is selected from Q and A,
or a naturally occurring variant sequence thereof;
or an antigenic fragment thereof.

Preferably, the isolated eukaryotic SSB protein or polypeptide is a mammalian SSB protein comprising the following amino acid sequence:

(SEQ ID NO: 10)

FX¹X²DX³KX^{B}GLKNLNX⁴X⁵FIVLEX⁶GRVTKTKDGHEVRX⁷CKVADX^{P}
TGSIX⁸ISVWDX⁹X¹⁰GX¹¹LIQX^{W}GDIIRLTX¹²GYASX¹³X¹⁴KGCLT
LYTGRGGX¹⁵LQKIGEFCMVYSEVPNESEPNPX¹⁶YX¹⁷X¹⁸QQ

wherein
X¹ is selected from V and I, X² is selected from K and R, X³ is selected from I and V, X^{B} is selected from P and A, X⁴ is selected from L and V, X⁵ is selected from I and V, X⁶ is selected from T and I, X⁷ is selected from T and S, X^{P} is selected from K and R, X⁸ is selected from N and T, X⁹ is selected from D and E, X¹⁰ is selected from V and L, X¹¹ is selected from N and G, X^{W} is selected from P and T, X¹² is selected from K and R, X¹³ is selected from V and M, X¹⁴ is selected from F and W, X¹⁵ is selected from D and E, X¹⁶ is selected from E and D, X¹⁷ is selected from S, R and N, and X¹⁸ is selected from T and G,
or a naturally occurring variant sequence thereof;
or an antigenic fragment thereof.

In a still further aspect, the present invention provides a polynucleotide molecule or oligonucleotide molecule comprising a nucleotide sequence encoding all or part (e.g. a biologically active fragment or antigenic fragment) of a eukaryotic SSB protein or polypeptide comprising an amino acid sequence as shown above as SEQ ID NO: 3 or SEQ ID NO: 10, and/or the complementary sequence thereto.

Such a polynucleotide molecule or oligonucleotide molecule may be used, for example, in the production of animal or cell line models of cancer which, in turn, might be used for screening cancer treatments and candidate anti-cancer agents. For example, an oligonucleotide molecule may be operably linked to a U6 or H1 RNA polymerase III promoter sequence, and introduced into a host (e.g. a recipient cell line or animal) to produce siRNA targeted to the relevant SSB gene, thereby generating a SSB-deficient or -depleted host.

The present invention further extends to an antibody or fragment thereof which specifically binds to a eukaryotic SSB protein or polypeptide comprising an amino acid sequence substantially corresponding to the amino acid sequence shown as SEQ ID NO: 4 or SEQ ID NO: 10, or a naturally occurring variant thereof. Still further, the present invention extends to a kit for diagnosing or prognosing cancer or a disposition to cancer, wherein the kit comprises any one or a combination of:

(i) an isolated eukaryotic SSB protein or polypeptide,
(ii) an antibody or fragment thereof according which specifically binds to a eukaryotic SSB protein or polypeptide, and
(iii) an oligonucleotide molecule suitable for use as a probe or primer sequence, comprising a nucleotide sequence encoding all or part of a eukaryotic SSB protein or polypeptide comprising an amino acid sequence as shown above as SEQ ID NO: 4 or SEQ ID NO: 10, and/or the complementary sequence thereto.

The present invention is hereinafter further described by way of the following, non-limiting examples and accompanying figures.

### EXAMPLES

### Example 1

### Identification and Characterisation of Novel Protein hSSB1

### Methods and Materials

Plasmids, recombinant protein purification, cell lines and siRNA GFP-hSSB1 fusion protein was expressed from pEGFPc1 as described previously (Pierce et al., 1999) and Rodrigue et al., 2006). Recombinant His-tagged hSSB1 was expressed from pET28c and pDEST17 respectively, in BL21 cells (Stratagene, La Jolla, Calif., United States of America). For purification of recombinant protein, BL21 cells were lysed in Ni A buffer (50 mM KCl, 50 mM KH₂PO₄, 10 mM imidazole, 20 mM β-mercaptoethanol, 10% w/v glycerol, 1 mg/ml lysozyme, 5 mM EDTA, and Complete Mini EDTA-free Protease inhibitor cocktail tablets). The resulting extract was diluted to 1 mM EDTA and passed over Qiagen Ni-NTA Superflow resin. The resin was washed with Ni A buffer and bound protein eluted in Ni B buffer (50 mM KCl, 50 mM KH₂PO₄, 100 mM imidazole, 20 mM β-mercaptoethanol, 10% w/v glycerol). The eluate was then passed over GE Healthcare HiTrap Heparin HP and washed with Buffer A (25 mM Tris pH 8.0, 100 mM NaCl, 1 mM DTT, and 10% w/v glycerol). Protein was then eluted in Buffer A containing 1 M NaCl. 1 ml of the most concentrated fraction was passed over a Superdex 200 column and fractions containing the protein aliquoted and stored at −80 degrees.

Small interfering RNAs (siRNA) were synthesised by Invitrogen (Invitrogen Corporation, Carlsbad, Calif., United States of America). The target sequences were hSSB1-GACAAAGGACGGGCATGAG (SEQ ID NO: 8), ATM-GCGCCTGATTCGAGATCCU (SEQ ID NO: 11) and control-UUCUCCGAACGUGUCACGU (SEQ ID NO: 12).

### Antibodies and Immunofluorescence

Antibodies were supplied by Calbiochem (Rad50, Mre11, Rad51), Upstate (γH2AX), Roche (BRDU), Cell Signalling Technologies (pT68-11 Chk2, pS317-Chk1, pS15-p53) and Invitrogen (Alexa secondary antibodies). Sheep antiserum to hSSB1 was raised against full-length recombinant His-tagged hSSB1 using standard methods. Rabbit antiserum was raised against a phosphorylated peptide representing the T117 hSSB1 phosphorylation site (i.e. NPEYSpTQQAPN; SEQ ID NO: 5). This antibody was used to detect hSSB1 by Western blotting and immunofluorescence.

For immunofluorescent staining, cells were pre-permeabilised with 20 mM HEPES, 120 mM KCl, 0.5% NP40 (w/v) for 15 min on ice prior to fixation in 4% paraformaldehyde (w/v) in phosphate buffered saline (PBS) for 10 minutes.

### Assays

MTT assays were performed 48 hrs following ionising radiation (IR) according to methods described by Slavotinek et al. (1994). G₁/S checkpoint was measured using the BrdUrd incorporation assay as described by Fabbro, 2004. For analysis of chromosomal aberrations at metaphase, exponentially growing cells were treated with 2 Gy of IR. Colcemid was added at various time points. Cells at metaphase were collected and chromosomal aberrations were scored as described previously (Pandita et al., 2006).

For MRN binding assays, protein complexes containing 50 ng of biotinylated NBS1 were incubated with Promega Streptavidin MagneSphere Paramagnetic Particles in buffer A (25 mM Tris pH 8.0, 100 mM NaCl, 1 mM DTT, 0.1% CHAPS, and 10% w/v glycerol) for 1 hr at room temperature. Beads were then isolated and placed in a fresh 1.5 ml microcentrifuge tube. 130 ng of hSSB1 in buffer A was incubated with the MRN bound beads for 30 minutes. The beads were washed three times with buffer A. Bound proteins were eluted with SDS loading buffer and immunoblotted with anti-hSSB1 antibodies. The appearance of ssDNA was detected using a BrdUrd incorporation assay by incubating cells with BrdUrd (10 ug/ml) for 30 hours as per Raderschall et al. (1999).

EMSA assays were conducted as previously described (Wadsworth et al., 2000).

### Results and Discussion

Database Mining for a Novel Single Stranded Binding Protein, hSSB1

Using the S. solfataricus SSB amino acid sequence, the human genome sequence was interrogated using the BLAST algorithm (NCBI http://www.ncbi.nlm.nih.gov/BLAST/). This revealed the presence of two highly conserved sequence homologues of S. solfataricus SSB (FIG. 1), present on chromosomes 2q13.3 and 2q32.3 respectively, which have been designated hSSB1 (i.e. human ssDNA binding protein 1) and hSSB2 (i.e. human ssDNA binding protein 2). Both proteins have a highly conserved N-terminal OB-fold domain, followed by a variable region with no predicted structure and a conserved C-terminal tail. Gel filtration data indicated that hSSB1 exists in a dimeric form in solution (data not shown). The database mining also revealed that homologues for both the hSSB1 and hSSB2 genes exist in other mammals, and single homologues were located in other divergent eukaryotic species (i.e. Xenopus laevis, Danio rerio and Drosophila melanogaster)(see FIG. 1).

hSSB1 Binding of ssDNA

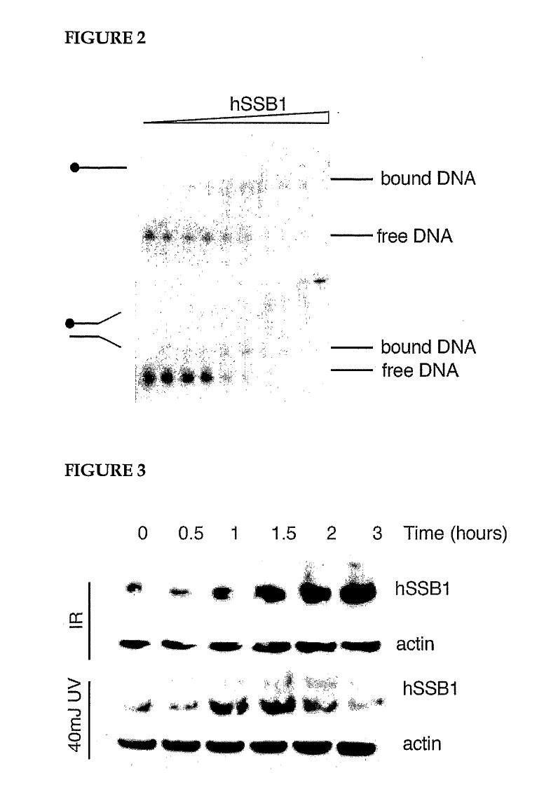

Recombinant hSSB1 cDNA was cloned to generate an N-terminal His tag. The resulting His-tagged recombinant hSSB1 was expressed in Escherichia coli. The capacity for this protein to bind ssDNA was confirmed in vitro by EMSA as shown in the upper lanes of FIG. 2. Further, the capacity for binding during replication was demonstrated by conducting assays in the presence of a synthetic replication fork (lower lanes of FIG. 2). These results confirm that hSSB1 functions as a DNA binding protein. Moreover, these results when considered in combination with the observation of structural similarities to existing SSB proteins, strongly indicates a role for hSSB1 in DNA replication and repair.

Overexpression of hSSB1 in Response to DNA Damage

To gain further insight into the function of hSSB1, polyclonal antibodies against hSSB1 were raised and affinity purified to investigate hSSB1 expression. In human neonatal foreskin fibroblasts (NFFs), the antibody recognised a band of approximately 36 kDa. The specificity of this protein was confirmed by pre-treatment with hSSB1-specific siRNA oligonucleotides and control siRNAs. The results showed diminished signal intensity in cells treated with hSSB1 specific siRNA oligonucleotides but not control siRNAs (data not shown).

To investigate the involvement of hSSB1 in the cellular response to DNA damage, NFFs were treated with different genotoxic agents, including IR and UV radiation. NFFs exposed to IR (6 Gy) or UV (20 mJ/m2) were extracted and hSSB1 was analysed by Western immunoblotting using affinity purified polyclonal anti-hSSB1 antibody. Cells were harvested at 0, 0.5, 1, 1.5, 2, and 3 hour time points. FIG. 3 shows the overexpression of hSSB1 in the presence of DNA damaging agents with a dose dependent response of hSSB1 to IR and UV. Following UV exposure, the characteristic dose dependent response appeared to cease after 1.5 hours, which is probably caused by DNA damage-induced impairment in cell function or cell death. These results indicate a role for hSSB1 in DNA replication or repair.

IR Sensitivity in hSSB1 Deficient Cells

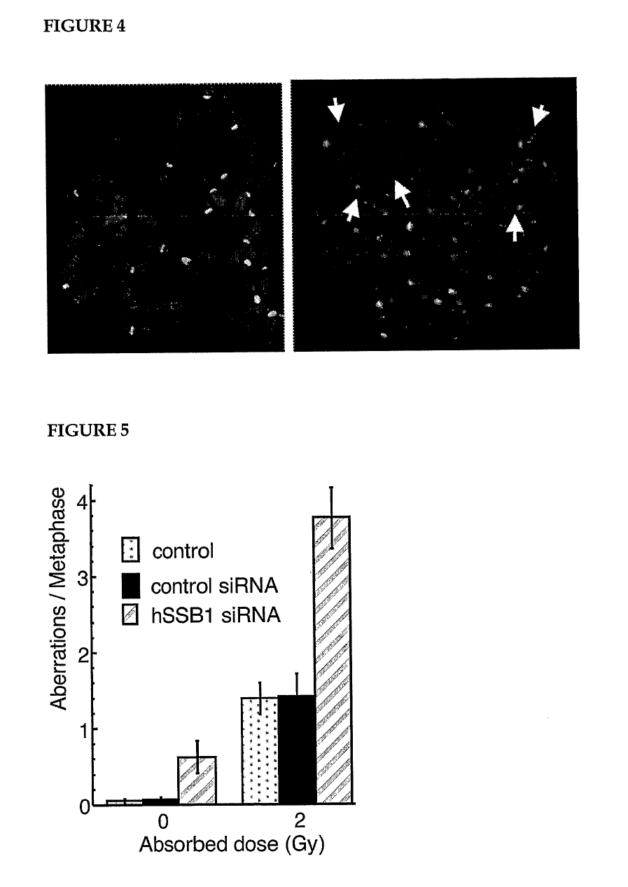

To assess the effect of suppressing hSSB1 function on the cellular response to DNA damage, NFFs were transfected with hSSB1-specific siRNA and control siRNA. Overall levels of hSSB1 were reduced by >90% compared to control cells and a substantial increase in IR-induced cell death was observed. Also, irradiated hSSB1-deficient cells at metaphase displayed a higher frequency of spontaneous chromosomal aberrations, which were rapidly accumulated (FIG. 4). These represented a statistically significant increase in the number of chromosomal aberrations (i.e. chromosome breaks, chromatid breaks and fragments thereof) compared with cells transfected with control siRNA.

FIG. 5 shows the frequencies of spontaneous and IR (2 Gy) induced chromosomal aberrations in control and hSSB1-deficient cells. Fifty metaphases for each sample were analysed for chromosomal aberrations, both chromatid and chromosomal aberrations were observed, in hSSB1-deficient cells. The results obtained were the mean of three independent experiments. The incidence of metaphase aberrations following IR was increased in hSSB1-deficient cells from approximately 1.4 aberrations in control cells to approximately 3.7 aberrations with hSSB1 specific siRNA. The accumulation of spontaneous DNA damage could also be observed in the absence of externally applied DNA damaging agents in the hSSB1-deficient control cells. These cells showed approximately 0.6 aberrations, while cells with functional hSSB1 showed almost no aberrations. Taken together, these results indicate that hSSB1 plays a functionally important role in allowing cells to repair genotoxic damage and maintain chromosome stability during the cell cycle.

hSSB1 Mediated Arrest of DNA Replication Following DNA Damage

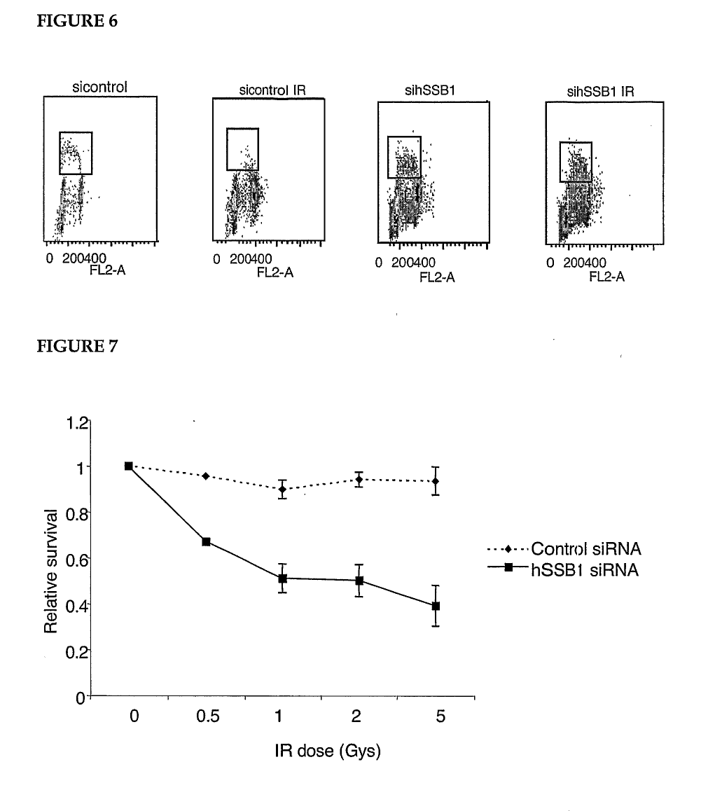

The integrity of cell cycle checkpoints in the NFF cells was also investigated. The G₁/S checkpoint was measured by staining cells with BrdUrd in the absence or presence of IR (Fabbro, M., (2004)). Cells were transfected with a control siRNA and hSSB1-specific siRNA and harvested 48 hrs later. Cells either remained untreated or were irradiated with 6 Gy IR and then incubated for 16 hrs before being pulsed for 30 min with BrdUrd (10 ug/ml). Cells were subsequently stained with anti-BrdUrd-FITC antibodies and propidium iodide and then analysed by flow cytometry. In control siRNA treated cells, there was a >50% reduction in BrdUrd incorporation after IR (FIG. 6, the boxed area indicates percentage of BrdU positive cells), illustrating an efficient arrest at the G₁/S checkpoint. Strikingly, BrdUrd incorporation was not significantly affected in hSSB1-deficient cells after IR, indicating a clear defect in the G₁/S checkpoint.

Dose Dependent Cell Death from DNA Damaging Agents

The functional consequences of treating human NFFs deficient in hSSB1 with IR were ascertained by MTT clonogenic survival assays. In these assays, NFF cells were treated with hSSB1 siRNA 48 hours prior to treatment with IR at 0, 0.5, 1, 2 and 5 Gys. Cells were then allowed to grow for a further 36 hours before rates of metabolism were measured by the MIT assay. Consistent with the chromosomal instability observed from metaphase aberrations, sensitivity to IR in hSSB1 deficient cells was reflected by a reduction in cell survival (FIG. 7). A dose dependent relationship was observed between IR dose and cell survival, indicating a direct relationship between DNA damage accrued in the absence of functional hSSB1, and cell death.

hSSB1 Localisation to Foci Following DNA Damage

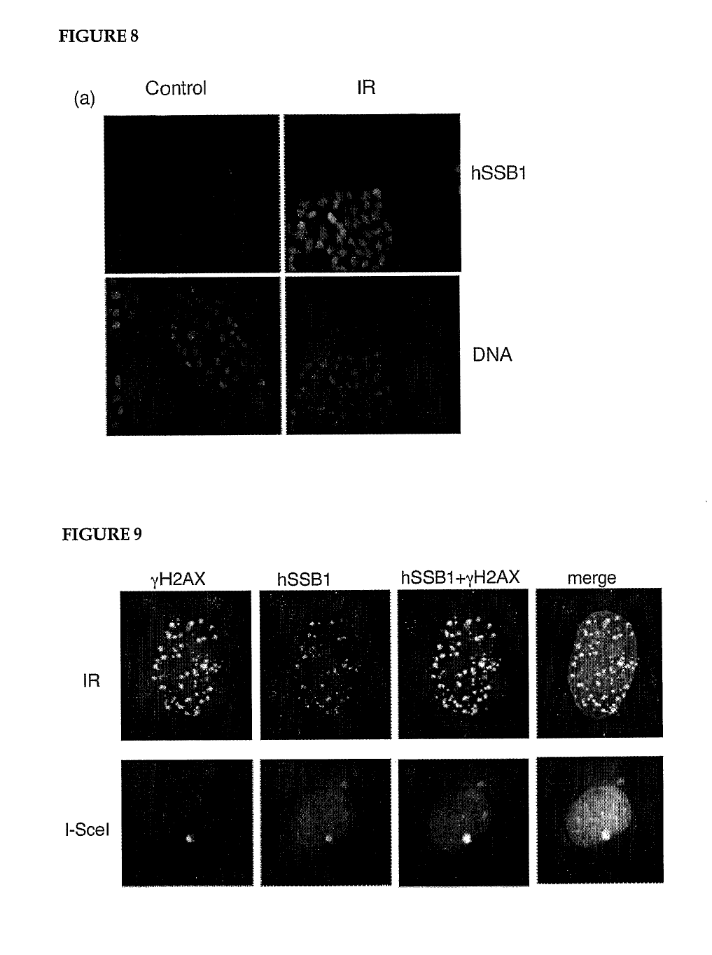

To further investigate the role of hSSB1 in the DNA damage response, immunofluorescence studies were carried out. In particular, NFFs with or without prior treatment with IR (6Gy) were extracted with detergent prior to permeabilisation, and then immunostained with anti-hSSB1 antibody for hSSB1 detection. Slides were viewed with a Bio-Rad confocal laser microscope. In FIG. 8, unirradiated cells showed weak nuclear staining with a rapid increase in hSSB1 levels evident within the nucleus following IR exposure. Pre-fixative detergent extraction revealed that hSSB1 becomes localised to prominent nuclear foci within 30 minutes of DNA damage (FIG. 9) with foci still present up to 8 hours later. Focus formation was also seen to be dose-dependent, with the average number of hSSB1 foci per cell increasing with IR dosage (data not shown). It is therefore clear that hSSB1 is rapidly recruited to DNA repair foci after DNA damage by IR, further indicating a role for the protein in DNA damage response.

hSSB1 Colocalises with γ-H2AX

It is known that phosphorylation of histone H2AX (γ-H2AX) is essential to the efficient recognition and repair of double strand breaks (DSBs) (i.e. H2AX becomes rapidly phosphorylated at the site of each nascent DSB; Burma et al., 2001). Further immunoflourescence studies were therefore carried out with a polyclonal antibody to γ-H2AX (Upstate Biotechnology) with the results viewed by sequential scanning of the two emission channels used (FIG. 9). hSSB1 foci showed striking co-localisation with foci formed by the phosphorylated H2AX complex (γ-H2AX). hSSB1 was also seen to be recruited to and co-localises with γ-H2AX at an I-Sce1 induced chromosomal double strand break. This shows a response by hSSB1 to DSBs that is analogous to γ-H2AX, possibly resulting from either a indirect or direct association with γ-H2AX.

hSSB1 Colocalises with MRN Proteins

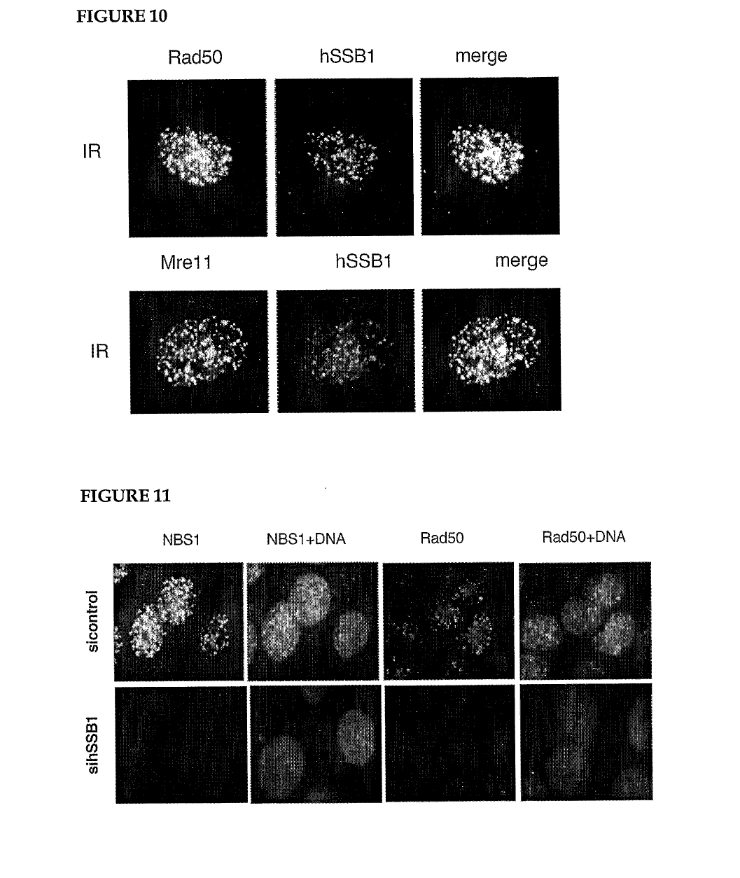

Recently, a short peptide motif in the C-terminus of Nijmegen Breakage protein (Nbs1), a component of the MRN complex, was shown to mediate recruitment of ATM to sites of DSBs, leading to the activation of ATM (Falck et al., 2005). The MRN complex also localises to nuclear foci upon DSB induction. To determine whether hSSB1 co-localises with the components of the MRN complex after DNA damage, localisation of hSSB1 and components of the MRN complex were examined in undamaged cells and in cells treated with IR. Co-localisation of hSSB1 with foci formed by Rad50 and Mre11 in NFF cells was analysed 1 hour after irradiation at 6 Gy. FIG. 10 shows that damage-induced hSSB1 clearly co-localises with Rad50 and Mre11 indicating that hSSB1 is required to recruit the MRN complex to foci and for resection of DSBs and HR repair.

Recruitment of DNA Damage Response Molecules by hSSB1

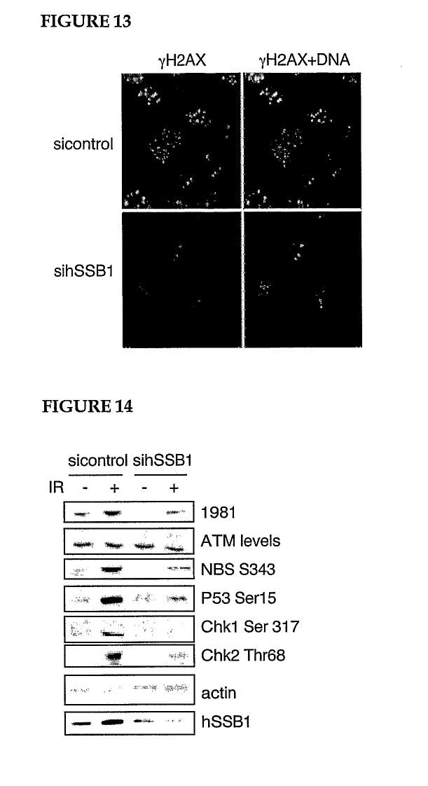

To test whether hSSB1 recruits the MRN complex and other proteins to foci, immunofluorescence studies were conducted with antibodies against NBS1 (Queensland Institute of Medical Research, Herston, QLD, Australia), Rad50 (Calbiochem), and γ-H2AX in NFFs transfected with hSSB1-specific siRNA and control siRNA. 48 hours after siRNA transfection, cells were irradiated and left to recover for 1 hour prior to fixation and immunostaining with anti-NBS1, anti-Rad50, anti-Rad51 and anti-γ-H2AX antibodies. This revealed that cells in which hSSB1 was depleted (i.e. cells treated with hSSB1-specific siRNA), were markedly impaired in their ability to form NBS1, Rad50, Rad51 and H2AX foci within 1 hour after IR (FIGS. 11, 12 and 13), whereas MRN and H2AX foci formed normally in control siRNA transfected cells. This shows that the MRN and H2AX response to DNA damage is dependent on hSSB1 activity. It was noted that abrogation of MRN and H2AX foci was not complete, suggesting that either the siRNA treatment leaves residual, functional hSSB1 capable of limited focus formation, or that a partly-redundant pathway exists to localise these proteins.

hSSB1 Initiates Cell Cycle Regulators

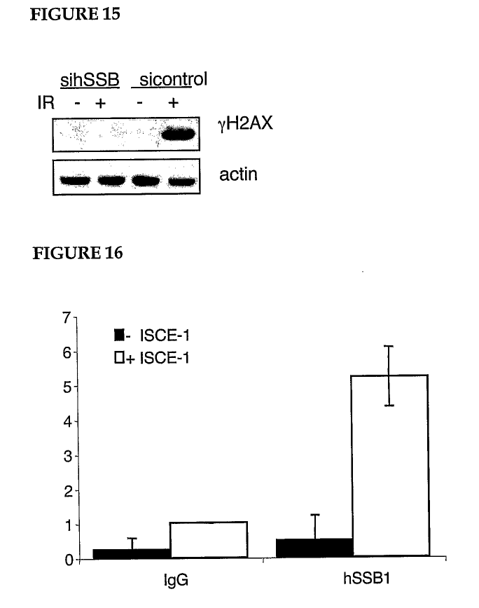

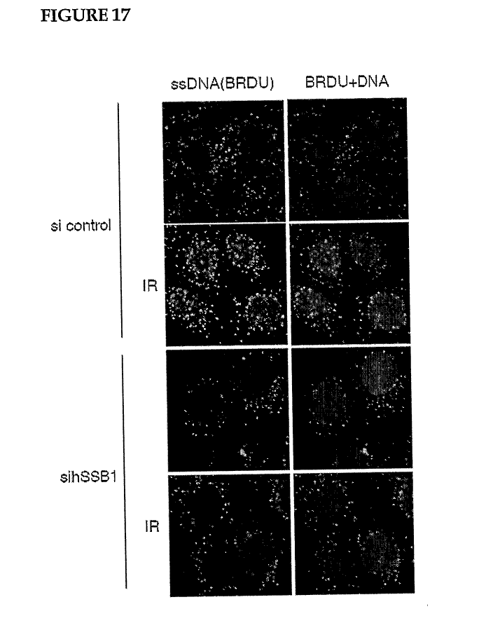

To gain insight into the mechanism by which hSSB1 mediates G₁/S damage activated cell cycle checkpoints, hSSB1 depleted NFF cells were assessed for their ability to phosphorylate key effector molecules known to be critical for efficient checkpoint activation after IR. That is, NFFs were transfected with hSSB1-specific siRNA or control siRNA, irradiated 48 hours later and left to recover for 30 minutes before cell extraction. Cell lysates were then immunoblotted with ATM (GeneTex, Inc., San Antonio, Tex., United States of America), NBS 5343 (Queensland Institute of Medical Research, Herston, QLD, Australia), p53 Ser15, Chk1 Ser317, Chk2 Thr68 (Cell Signalling Technologies), γ-H2AX antibodies (Upstate Biotechnology) and control antibodies for actin (Sigma) and hSSB1. As expected, the irradiation of cells expressing the control siRNA led to the autophosphorylation of ATM and phosphorylation of the ATM targets p53, Chk1, Chk2, NBS1 and γ-H2AX (FIGS. 14 and 15). However, exposure of parallel cultures of NFFs transfected with hSSB1-specific siRNA to IR did not induce a similar degree of phosphorylation of these proteins indicating that hSSB1 is required for DNA damage induced activation of ATM and for the phosphorylation of downstream targets. These results indicate that the damage response in cells deficient in hSSB1 is impaired, implicating hSSB1 as a critical regulator of the DNA damage response pathway.

hSSB1 Localised to Double Stranded Breaks

In order to study hSSB1 recruitment for repair of a single lesion, the MCF7 cells with stably integrated pDR-GFP plasmid, DR-GFP (Pierce et al., 1999) was used. This cell line contains a stably-integrated plasmid with a modified GFP gene in which an I-SceI cleavage site has been engineered, such that a unique DSB can be created in a known nucleotide sequence. Following transfection of MCF7DR-GFP with the I-SceI plasmid, a single focus of hSSB1 was visible which was not apparent in the absence of I-SceI expression. As previously discussed, this focus co-localised with γH2AX. Real-time PCR on chromatin immunoprecipitation (ChIP) samples was carried out using primers directed at 94-378 nucleotides from the DSB. The enrichment of hSSB1 following induction of the DSB was compared to that of an IgG control (normalised with an internal control towards a locus elsewhere in the genome) to provide the increase in enrichment relative to baseline (FIG. 16). The experiment was repeated three times and PCR reactions were performed in duplicate on each occasion. ChIP revealed that hSSB1 binds between 94 and 378 by to the I-SceI induced DSB in vivo. This relative proximity suggests that hSSB1 has a direct role in repairing DNA.

hSSB1 in Homologous Recombination Repair

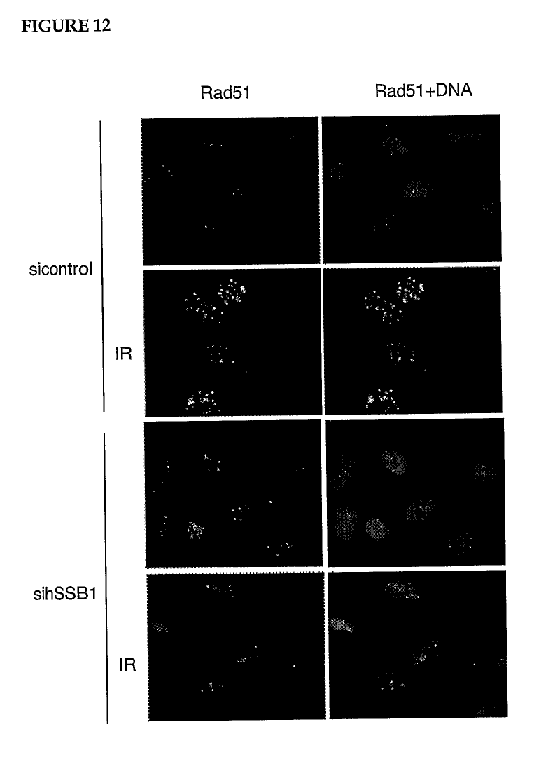

From previous studies, it was known that hSSB1 and Rad51 do not interact directly (data not shown), therefore the observed reduction in Rad51 foci formation by hSSB1-specific siRNA mediated down-regulation of hSSB1 was unexpected (FIG. 12). It was reasoned that the defect in Rad51 foci formation might be due to a defect in the generation of ssDNA formed after resection of DSBs. The ssDNA/Rad51 nucleoprotein filament mediates homology searches and invades intact homologous duplex DNA to form Holliday junction recombination intermediates, before branch migration and resolution restores the broken DNA sequence. Accordingly, the appearance of ssDNA was studied using a BrdUrd incorporation assay. 24 hours after siRNA-transfection, cells were incubated with BrdUrd (10 ug/ml) for 30 hours and stained to visualise ssDNA following irradiation (6 Gy). In response to IR, 33% of control siRNA treated cells showed BrdUrd foci formation whereas most of the hSSB1-depleted cells did not exhibit ssDNA foci formation (FIG. 4e). These findings indicate a possible defect in homologous recombination (HR) repair in hSSB1-deficient cells, since the generation of ssDNA after DNA damage is a prerequisite for this type of repair.

hSSB1 is Overexpressed in Homologous Recombination Repair

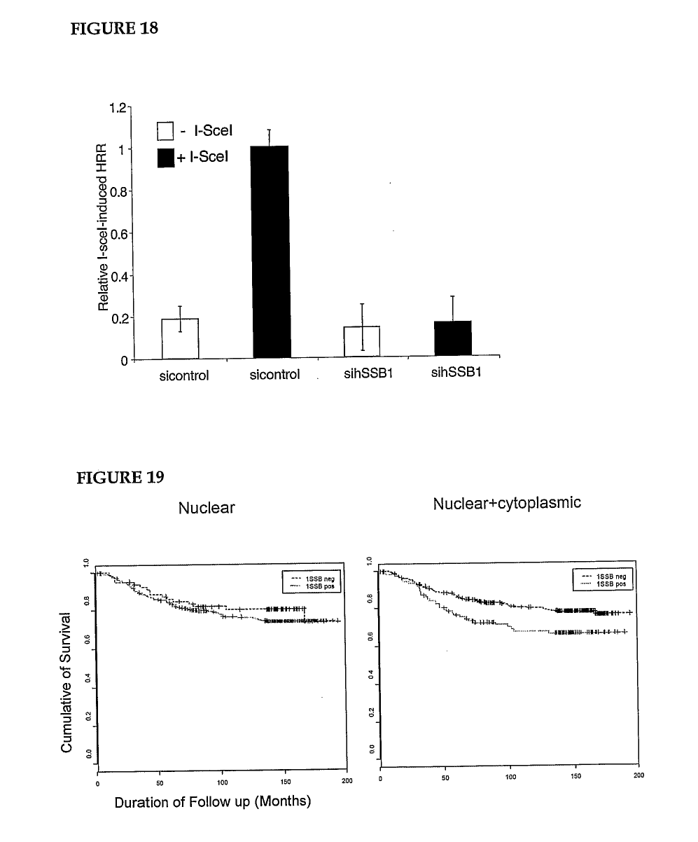

In order to quantify hSSB1 induced HR repair, reconstitution of a green fluorescent protein reporter gene (pDR-GFP) within a chromosomally integrated plasmid substrate in cells with or without the silencing of hSSB1 gene expression, was assayed as described previously (Pierce et al., 1999 and Zhang, et al., 2005). To detect HR repair of an induced chromosomal DSB, the I-SceI expression vector (pCBSCE) was transfected transiently into MCF7 cells containing a stably-integrated pDR-GFP plasmid (MCF7 DRGFP cells) 24 hours after siRNA transfection. 48 hours after pCBSCE transfection, FACS analysis was carried out to quantify GFP positive cells. The results shown in FIG. 18 are the average of three independent experiments and error bars indicate the standard deviation.

Treatment of I-SceI positive MCF7 DRGFP cells with the hSSB1 specific siRNA reduced the number GFP positive cells (i.e. the relative homologous recombination repair events), compared to treatment with control siRNA (FIG. 18). This decrease was not attributed to the differences in transient transfection expression frequencies between cells, as the number of GFP positive cells obtained after transfection with pEGFP, containing the full-length cDNA of GFP, were comparable in hSSB1-depleted and control cells. Taken together, the results show that hSSB1 performs an early role in the initiation of HR by promoting efficient resection of DSBs. The resection defect in the absence of hSSB1 may, in part, be due to a failure to recruit the MRN complex to sites of DSBs. However, it is currently unknown whether the MRN complex provides the nucleolytic activity required for DSB processing. It is also thought that unidentified nucleases other than Mre11 may also participate in DSB resection in mitotic cells (Tsubouchi et al., 2000). Alternatively, hSSB1 may be required to maintain the stability of generated ssDNA ends.

### Example 2

### hSSB1 Expression as a Marker for Tumours, Cancers and Cancer Predisposition

The work and results described in Example 1 clearly demonstrate that hSSB1 is the central component of the homologous DNA repair pathway responsible for repairing double stranded DNA breaks. As shown, the loss of hSSB1 in primary fibroblasts results in the loss of the cell's ability to initiate DNA damage signalling pathways and initiate homologous recombination repair following exposure to DNA damaging agents. This, in turn, results in chromosomal instability, the accumulation of spontaneous mutation and eventually cell death. As chromosomal aberrations are observed at G₁/S phases of cell replication (FIG. 3), these aberrations are inherited in daughter cells and have the potential to metastisise in vivo. Accordingly, experimentation was undertaken to investigate cells transformating from normal cells to tumours to determine whether the observed expression patterns differ in normal, pre-tumour and tumour cells.

### Methods and Materials

hSSB1 Expression During Cellular Transformation

MCF10A series of cell lines were obtained from Barbara Ann Karmanos Cancer Institute (Detroit, Mich., United States of America). It consists of immortal MCF10A line (from a woman with fibrocystic disease, transformed MCF10AT (MCF10A transfected with T24 Ha-ras) with potential for neoplastic progression, and a fully malignant MCF10CA. Tumour and pre-tumour cells were subsequently assayed for hSSB1 expression by Western immunoblotting using the affinity purified polyclonal anti-hSSB1 antibody described in Example 1.

hSSB1 Function in Tumour Cell Lines

300 breast tumour and 140 bowel cancer tissue samples were obtained from Professor Lakhani (Medical School, Herston, QLD, Australia) and Professor Leggett (Royal Brisbane Hospital, QLD, Australia). hSSB1 expression in these samples was assessed by staining tissue sections with the polyclonal anti-hSSB1 antibody described in Example 1 using standard methods.

### Results and Discussion

hSSB1 Expression During Cellular Transformation

Tert-immortalised mammary epithelial cells (MEC), or spontaneously immortalised MEC from fibrosarcoma patients, show a very low level of SSB1 expression. Expression is still low in pre-malignant Ras-transformed cells, however malignant MEC which form tumours in mice, showed significantly elevated levels of hSSB1 expression (data not shown).

hSSB1 Function in Tumour Cell Lines

The functional consequences of hSSB1 suppression in cancer cell lines, HeLa (cervical cancer) and 293T (kidney cancer) were investigated by transfection with hSSB1-specific siRNA and control siRNA. While hSSB1-specific siRNA was not fatal to control cells (as shown above), HeLa and 293T cells could not tolerate hSSB1 deficiency during normal growth conditions, rapidly entering into apoptosis. In these cell lines, hSSB1 is overexpressed with respect to hSSB1 deficient NFF cells. As cancer is well known to initiate chromosomal rearrangements, the observed differences between primary cells and cancer cells may result from a greater frequency of endogenous DNA damage events occurring within cancer cells. Alternatively, they may result from the inability to repair DNA damaged caused by normal cellular processes and oxidative stress. This, in addition to the loss of hSSB1 and hence the ability to initiate DNA damage signalling pathways, is a likely cause of rapid cell death.

In the light of these findings, hSSB1 expression was studied in over 300 breast tumour and about 140 bowel cancer tissue samples, taken from historical tissue collections, and compared with the patient history to determine the effectiveness of hSSB1 as a diagnostic and prognostic marker. Tissue samples were stained for hSSB1 and assessed by a consultant pathologist. Over 80% of the tumours were independently classed as hSSB1 positive and, as shown in FIG. 19, those tissue samples showing hSSB1 expression following staining indicated a poorer prognostic outcome in patients, in comparison with patients not showing positive hSSB1 staining. Further, the prognostic outcome of patients producing tissue samples showing both nuclear and cytoplasmic staining for hSSB1 was poorer than that of patients positive for hSSB1 staining in the cell nucleus only.

Intracellular Localisation of hSSB1 Expression

As shown about, the results obtained from all of the screened breast tumour tissue samples were correlated with patient pathology data which showed expression rates to be statistically linked to patient prognosis. Further statistical analysis indicated that the intracellular location of hSSB1 expression may also act as a predictor of patient outcome, therefore demonstrating considerable potential for use as a prognostic tool. That is, in some of the tumour tissue samples, cytoplasmic as well as nuclear staining was observed for hSSB1, and this appears to correlate with a worse patient survival outcome than detection of nuclear staining alone. While not wishing to be bound by theory, it is considered that tumours showing cytoplasmic staining may represent cells with much higher levels of hSSB1 expression, therefore accounting for the prognostic potential of cellular staining.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Burma, S., et al., ATM phosphorylates histone H2AX in response to DNA double-strand breaks. J Biol Chem. 276:42462-424627 (2001).
2. Fabbro, M., BRCA1-BARD1 complexes are required for p53Ser-15 phosphorylation and a G₁/S arrest following ionizing radiation-induced DNA damage. J Biol Chem. 279:31251-31258 (2004).
3. Falck, J., et al., Conserved modes of recruitment of ATM, ATR and DNA-PKcs to sites of DNA damage. Nature. 434:605-611 (2005).
4. Pandita, R., et al., Mammalian Rad9 plays a role in telomere stability, Sand G2-phase-specific cell survival, and homologous recombinational repair. Mol Cell Biol. 26:1850-64 (2006).
5. Paull, T. T., Rogakou, E. P., Yamazaki, V., Kirchgessner, C. U., Gellert, M., Bonner, W. M., A critical role for histone H2AX in recruitment of repair factors to nuclear foci after DNA damage Curr Biol. 10(15):886-895 (2000).
6. Pierce, A. J., et al., XRCC3 promotes homology-directed repair of DNA damage in mammalian cells. Genes Dev. 13:2633-2638 (1999).
7. Raderschall, E., et al., Nuclear foci of mammalian recombination proteins are located at single-stranded DNA regions formed after DNA damage. Proc Natl Acad Sci USA. 96:1921-1926 (1999).
8. Rodrigue, A., et al., Interplay between human DNA repair proteins at a unique double-strand break in vivo. EMBO J. 25:222-31 (2006).
9. Slavotinek, A., et al., Measurement of radiation survival using the MTT assay. European J Cancer 30:1376-1382 (1994).
10. Tsubouchi, H. and Ogawa, H., Exo1 roles for repair of DNA double-strand breaks and meiotic crossing over in Saccharomyces cerevisiae. Mol Biol Cell. 11:2221-2233 (2000).
11. Wadsworth, R. I. M. and White, M. F., Identification and properties of the crenarchaeal single-stranded DNA binding protein from Sulfolobus solfataricus. Nucleic Acids Res., 29:914-920 (2001).
12. Zhang, J., et al., MDC1 interacts with Rad51 and facilitates homologous recombination. Nat Struct Mol Biol. 12:902-909 (2005).
13. Zhou, B. B. and Elledge, S. J., The DNA damage response: putting checkpoints in perspective. Nature. 408:433-439 (2000).

1. A method of detecting transformed cells or tumour cells comprising the step of detecting in a suitable biological sample, overexpression of a human ssDNA binding (SSB) protein or polypeptide comprising the following amino acid sequence:
(SEQ ID NO: 1)
FX¹X²DX³KPGLKNLNX⁴X⁵FIVLEX⁶GRVTKTKDGHEVRX⁷CKVA
DKTGSIX⁸ISVWDX⁹X¹⁰GX¹¹LIQPGDIIRLTX¹²GYASX¹³X¹⁴KG
CLTLYTGRGGX¹⁵LQKIGEFCMVYSEVPNFSEPNPX¹⁶YX¹⁷X¹⁸QQ
wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof.

2. A method of diagnosing or prognosing cancer or assessing a predisposition to cancer, said method comprising the step of detecting in a suitable biological sample from a subject, overexpression of a human SSB protein or polypeptide comprising the following amino acid sequence:
(SEQ ID NO: 1)
FX¹X²DX³KPGLKNLNX⁴X⁵FIVLEX⁶GRVTKTKDGHEVRX⁷CKV
ADKTGSIX⁸ISVWDX⁹X¹⁰GX¹¹LIQPGDIIRLTX¹²GYASX¹³X¹⁴KG
CLTLYTGRGGX¹⁵LQKIGEFCMVYSEVPNFSEPNPX¹⁶YX¹⁷X¹⁸QQ
wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof.

3. The method of claim 1, wherein said biological sample is a tissue biopsy, a blood sample or a faecal sample.

4. The method according to claim 1, wherein the cancer is a breast cancer or bowel cancer.

5. The method according to claim 1, wherein said method is used for selecting a suitable treatment for cancer or for assessing the effectiveness of a cancer treatment.

6. A method according to claim 1, wherein said SSB protein or polypeptide is a human SSB1 protein or polypeptide comprising an amino acid sequence substantially corresponding to the following:
(SEQ ID NO: 2)
MTTETFVKDIKPGLKNLNLIFIVLETGRVTKTKDGHEVRTCKVADKTGS
INISVWDDVGNLIQPGDIIRLTKGYASVFKGCLTLYTGRGGDLQKIGEFC
MVYSEVPNFSEPNPEYSTQQAPNKAVQNDSNPSASQPTTGPSAASP
ASENQNGNGLSAPPGPGGGPHPPHTPSHPPSTRITRSQPNHTPAGPP
GPSSNPVSNGKETRRSSKR,
or a naturally occurring variant sequence thereof.

7. A method according to claim 1, wherein said step of detecting overexpression of said SSB protein or polypeptide comprises;
(i) determining the relative amount of messenger RNA encoding the protein or polypeptide that is present in said sample,
(ii) determining the relative amount, in said sample, of an antibody or a fragment thereof that specifically binds to said SSB protein or polypeptide, or
(iii) determining the relative amount of the protein or polypeptide or a fragment thereof that is present in the said sample.

8. A method according to claim 1, wherein said step of detecting overexpression of said SSB protein or polypeptide comprises determining the relative amount of the protein or polypeptide or a fragment thereof that is present in the said sample.

9. An isolated eukaryotic SSB protein or polypeptide comprising the following amino acid sequence:
(SEQ ID NO: 3)
X^{A}X¹X²DX³KX^{B}GX^{C}KNX^{D}X^{E}X⁴X⁵FIVLEX⁶GX^{F}X^{G}TX^{H}TKX^{I}X^{J}X^{K}EV
RX⁷X^{L}X^{M}VX^{N}DX^{O}X^{P}X^{Q}X^{R}IX⁸X^{S}SX^{T}WDX⁹X¹⁰GX¹¹X^{U}IX^{V}X^{W}GDI
X^{X}RLTX¹²GYASX¹³X¹⁴X^{Y}X^{Z}CLTLYX^{AB}GX^{AC}X^{AD}GX¹⁵X^{AE}X^{AF}KI
GEX^{AG}CMVX^{AH}X^{AI}EX^{AJ}X^{AK}NX^{AL}SEPX^{AM}X^{AN}X¹⁶X^{AO}X¹⁷X¹⁸QX^{AP}
wherein
X^{A} is selected from F, L and P, X¹ is selected from V and I, X² is selected from K and R, X³ is selected from I and V, X^{B} is selected from P and A, X^{C} is selected from L and S, X^{D} is selected from L and I, X^{E} is selected from N and S, X⁴ is selected from L, V and I, X⁵ is selected from I, L and V, X⁶ is selected from T, I and V, X^{F} is selected from R and V, X^{G} is selected from V and A, X^{H} is selected from K and V, X^{I} is selected from D and E, X^{J} is selected from G and N, X^{K} is selected from H and R, X⁷ is selected from T, S and N, X^{L} is selected from C and F, X^{M} is selected from K and R, X^{N} is selected from A and G, X^{O} is selected from K, R and P, X^{P} is selected from T and S, X^{Q} is selected from G and A, X^{R} is selected from S and C, X⁸ is selected from N, T and A, X^{S} is selected from I and V, X^{T} is selected from V and I, X⁹ is selected from D and E, X¹⁰ is selected from V, I, L and P, X¹¹ is selected from N, G, S and K, X^{U} is selected from L and F, X^{V} is selected from Q and A, X^{W} is selected from P and T, X^{X} is selected from I and V, X¹² is selected from K and R, X¹³ is selected from V, M, L and I, X¹⁴ is selected from F and W, X^{Y} is selected from K and R, X^{Z} is selected from G and H, X^{AB} is selected from T and S, X^{AC} is selected from R and K, X^{AD} is selected from G and N, X¹⁵ is selected from D and E, X^{AE} is selected from L and V, X^{AF} is selected from Q and F, X^{AG} is selected from F and Y, X^{AH} is selected from Y and F, X^{AI} is selected from S and N, X^{AJ} is selected from V and S, X^{AK} is selected from P and V, X^{AL} is selected from F and M, X^{AM} is selected from N and K, X^{AN} is P or is null, X¹⁶ is selected from E and D or is null, X^{AO} is selected from Y, L and R, X¹⁷ is selected from S, R, N, I, L and A, X¹⁸ is selected from T, G, A and E, and X^{AP} is selected from Q and A,
or a naturally occurring variant sequence thereof;
or an antigenic fragment thereof.

10. The SSB protein or polypeptide of claim 9 comprising the following amino acid sequence:
(SEQ ID NO: 1)
FX¹X²DX³KPGLKNLNX⁴X⁵FIVLEX⁶GRVTKTKDGHEVRX⁷CKVADKT
GSIX⁸ISVWDX⁹X¹⁰GX¹¹LIQPGDIIRLTX¹²GYASX¹³X¹⁴KGCLTL
YTGRGGX¹⁵LQKIGEFCMVYSEVPNFSEPNPX¹⁶YX¹⁷X¹⁸QQ
wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof;
or an antigenic fragment thereof.

11. The SSB protein or polypeptide of claim 9 comprising an amino acid sequence substantially corresponding to the following:
(SEQ ID NO: 2)
MTTETFVKDIKPGLKNLNLIFIVLETGRVTKTKDGHEVRTCKVADKTGS
INISVWDDVGNLIQPGDIIRLTKGYASVFKGCLTLYTGRGGDLQKIGEF
CMVYSEVPNFSEPNPEYSTQQAPNKAVQNDSNPSASQPTTGPSAASP
ASENQNGNGLSAPPGPGGGPHPPHTPSHPPSTRITRSQPNHTPAGPPGP
SSNPVSNGKETRRSSKR,
or a naturally occurring variant sequence thereof.

12. The SSB protein or polypeptide of claim 9 comprising an amino acid sequence substantially corresponding to the following:
(SEQ ID NO: 4)
MNRVNDPLIFIRDIKPGLKNLNVVFIVLEIGRVTKTKDGHEVRSCKVAD
KTGSITISVWDEIGGLIQPGDIIRLTRGYASMWKGCLTLYTGRGGELQK
IGEFCMVYSEVPNFSEPNPDYRGQQNKGAQSEQKNNSMNSNMGTG
TFGPVGNGVHTGPESREHQFSHAGRSNGRGLINPQLQGTASNQTV;
or a naturally occurring variant sequence thereof.

13. The SSB protein or polypeptide of claim 9 comprising the following amino acid sequence:
(SEQ ID NO: 10)
FX¹X²DX³KX^{B}GLKNLNX⁴X⁵FIVLEX⁶GRVTKTKDGHEVRX⁷CKV
ADX^{P}TGSIX⁸ISVWDX⁹X¹⁰GX¹¹LIQX^{W}GDIIRLTX¹²GYASX¹³X¹⁴K
GCLTLYTGRGGX¹⁵LQKIGEFCMVYSEVPNFSEPNPX¹⁶YX¹⁷X¹⁸QQ
wherein
X¹ is selected from V and I, X² is selected from K and R, X³ is selected from I and V, X^{B} is selected from P and A, X⁴ is selected from L and V, X⁵ is selected from I and V, X⁶ is selected from T and I, X⁷ is selected from T and S, X^{P} is selected from K and R, X⁸ is selected from N and T, X⁹ is selected from D and E, X¹⁰ is selected from V and L, X¹¹ is selected from N and G, X^{W} is selected from P and T, X¹² is selected from K and R, X¹³ is selected from V and M, X¹⁴ is selected from F and W, X¹⁵ is selected from D and E, X¹⁶ is selected from E and D, X¹⁷ is selected from S, R and N, and X¹⁸ is selected from T and G,
or a naturally occurring variant sequence thereof;
or an antigenic fragment thereof.

14. An isolated antibody or fragment thereof which specifically binds to a human SSB protein or polypeptide comprising the following amino acid sequence:
(SEQ ID NO: 1)
FX¹X²DX³KPGLKNLNX⁴X⁵FIVLEX⁶GRVTKTKDGHEVRX⁷CK
VADKTGSIX⁸ISVWDX⁹X¹⁰GX¹¹LIQPGDIIRLTX¹²GYASX¹³X¹⁴K
GCLTLYTGRGGX¹⁵LQKIGEFCMVYSEVPNFSEPNPX¹⁶YX¹⁷X¹⁸QQ
wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof
or an antigenic fragment thereof.

15. The antibody or fragment thereof of claim 14, wherein said SSB protein or polypeptide is a human SSB1 protein or polypeptide comprising an amino acid sequence substantially corresponding to the following:
(SEQ ID NO: 2)
MTTETFVKDIKPGLKNLNLIFIVLETGRVTKTKDGHEVRTCKVADKTGS
INISVWDDVGNLIQPGDIIRLTKGYASVFKGCLTLYTGRGGDLQKIGEFC
MVYSEVPNFSEPNPEYSTQQAPNKAVQNDSNPSASQPTTGPSAASP
ASENQNGNGLSAPPGPGGGPHPPHTPSHPPSTRITRSQPNHTPAGP
PGPSSNPVSNGKETRRSSKR,
or a naturally occurring variant sequence thereof.

16. The antibody or fragment thereof of claim 14, wherein the antibody or fragment thereof specifically binds to an antigenic fragment of a human SSB1 protein or polypeptide, said antigenic fragment comprising an amino acid sequence substantially corresponding to the following:

| | |
|---|---|
| NPEYSTQQAPN | (SEQ ID NO: 5) |

17. The antibody or fragment thereof of claim 14, wherein said SSB protein or polypeptide is a human SSB2 protein or polypeptide comprising an amino acid sequence substantially corresponding to the following:
(SEQ ID NO: 4)
MNRVNDPLIFIRDIKPGLKNLNVVFIVLEIGRVTKTKDGHEVRSCKVAD
KTGSITISVWDEIGGLIQPGDIIRLTRGYASMWKGCLTLYTGRGGELQK
IGEFCMVYSEVPNFSEPNPDYRGQQNKGAQSEQKNNSMNSNMGTGT
FGPVGNGVHTGPESREHQFSHAGRSNGRGLINPQLQGTASNQTV;
or a naturally occurring variant sequence thereof.

18. An isolated polynucleotide or oligonucleotide molecule comprising a nucleotide sequence encoding all or part of a eukaryotic SSB protein or polypeptide comprising the following amino acid sequence:
(SEQ ID NO: 3)
X^{A}X¹X²DX³KX^{B}GX^{C}KNX^{D}X^{E}X⁴X⁵FIVLEX⁶GX^{F}X^{G}TX^{H}TK
X^{I}X^{J}X^{K}EVRX⁷X^{L}X^{M}VX^{NDX}^{O}X^{P}X^{Q}X^{R}IX⁸X^{S}SX^{T}WD
X⁹X¹⁰GX¹¹X^{U}IX^{V}X^{W}GDIX^{X}RLTX¹²GYASX¹³X¹⁴X^{Y}X^{Z}CL
TLYX^{AB}GX^{AC}X^{AD}GX¹⁵X^{AE}X^{AF}KIGEX^{AG}CMVX^{AH}X^{AI}EX^{AJ}X^{AK}N
X^{AL}SEPX^{AM}X^{AN}X¹⁶X^{AO}X¹⁷X¹⁸QX^{AP}
wherein
X^{A} is selected from F, L and P, X¹ is selected from V and I, X² is selected from K and R, X³ is selected from I and V, X^{B} is selected from P and A, X^{C} is selected from L and S, X^{D} is selected from L and I, X^{E} is selected from N and S, X⁴ is selected from L, V and I, X⁵ is selected from I, L and V, X⁶ is selected from T, I and V, X^{F} is selected from R and V, X^{G} is selected from V and A, X^{H} is selected from K and V, X^{I} is selected from D and E, X^{J} is selected from G and N, X^{K} is selected from H and R, X⁷ is selected from T, S and N, X^{L} is selected from C and F, X^{M} is selected from K and R, X^{N} is selected from A and G, X^{O} is selected from K, R and P, X^{P} is selected from T and S, X^{Q} is selected from G and A, X^{R} is selected from S and C, X⁸ is selected from N, T and A, X^{S} is selected from I and V, X^{T} is selected from V and I, X⁹ is selected from D and E, X¹⁰ is selected from V, I, L and P, X¹¹ is selected from N, G, S and K, X^{U} is selected from L and F, X^{V} is selected from Q and A, X^{W} is selected from P and T, X^{X} is selected from I and V, X¹² is selected from K and R, X¹³ is selected from V, M, L and I, X¹⁴ is selected from F and W, X^{Y} is selected from K and R, X^{Z} is selected from G and H, X^{AB} is selected from T and S, X^{AC} is selected from R and K, X^{AD} is selected from G and N, X¹⁵ is selected from D and E, X^{AE} is selected from L and V, X^{AF} is selected from Q and F, X^{AG} is selected from F and Y, X^{AH} is selected from Y and F, X^{AI} is selected from S and N, X^{AJ} is selected from V and S, X^{AK} is selected from P and V, X^{AL} is selected from F and M, X^{AM} is selected from N and K, X^{AN} is P or is null, X¹⁶ is selected from E and D or is null, X^{AO} is selected from Y, L and R, X¹⁷ is selected from S, R, N, I, L and A, X¹⁸ is selected from T, G, A and E, and X^{AP} is selected from Q and A,
or a naturally occurring variant sequence thereof;
and/or the complementary sequence thereto.

19. A polynucleotide molecule according to claim 18, wherein the polynucleotide molecule encodes a SSB protein or polypeptide comprising the following amino acid sequence:
(SEQ ID NO: 1)
FX¹X²DX³KPGLKNLNX⁴X⁵FIVLEX⁶GRVTKTKDGHEVRX⁷C
KVADKTGSIX⁸ISVWDX⁹X¹⁰GX¹¹LIQPGDIIRLTX¹²GYASX¹³X¹⁴K
GCLTLYTGRGGX¹⁵LQKIGEFCMVYSEVPNFSEPNPX¹⁶YX¹⁷X¹⁸QQ
wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof.

20. An oligonucleotide molecule according to claim 18, wherein the oligonucleotide molecule is suitable for use as a probe or primer sequence which hybridises under high stringency conditions to a polynucleotide molecule encoding a SSB protein or polypeptide comprising the following amino acid sequence:
(SEQ ID NO: 1)
FX¹X²DX³KPGLKNLNX⁴X⁵FIVLEX⁶GRVTKTKDGHEVRX⁷C
KVADKTGSIX⁸ISVWDX⁹X¹⁰GX¹¹LIQPGDIIRLTX¹²GYASX¹³X¹⁴K
GCLTLYTGRGGX¹⁵LQKIGEFCMVYSEVPNFSEPNPX¹⁶YX¹⁷X¹⁸QQ
wherein
X¹ is selected from V and I, X² is selected from K and R, X³ is selected from I and V, X⁴ is selected from L and V, X⁵ is selected from I and V, X⁶ is selected from T and I, X⁷ is selected from T and S, X⁸ is selected from N and T, X⁹ is selected from D and E, X¹⁰ is selected from V and I, X¹¹ is selected from N and G, X¹² is selected from K and R, X¹³ is selected from V and M, X¹⁴ is selected from F and W, X¹⁵ is selected from D and E, X¹⁶ is selected from E and D, X¹⁷ is selected from S and R, and X¹⁸ is selected from T and G,
or a naturally occurring variant sequence thereof
and/or the complementary sequence thereto.

21. A kit for diagnosing or prognosing cancer or assessing a predisposition to cancer, wherein said kit comprises
an isolated eukaryotic SSB protein or polypeptide according to claim 9.

22. A kit for diagnosing or prognosing cancer or assessing a predisposition to cancer, wherein said kit comprises an isolated antibody or fragment thereof according to claim 14.

23. A kit for diagnosing or prognosing cancer or assessing a predisposition to cancer, wherein said kit comprises an isolated polynucleotide molecule or oligonucleotide molecule according to any claim 18.


SEQUENCE LISTING (20 sequences)

SEQ ID NO: 1 - protein, 114 aa, Homo sapiens
  VARIANT (2)..(2): Xaa is selected from  Val and Ile
FXXDXKPGLKNLNXXFIVLEXGRVTKTKDGHEVRXCKVADKTGSIXISVWDXXGXLIQPGDIIRLTXGYASXXKGCLTLYTGRGGXLQKIGEFCMVYSEVPNFSEPNPXYXXQQ

SEQ ID NO: 2 - protein, 211 aa, Homo sapiens
MTTETFVKDIKPGLKNLNLIFIVLETGRVTKTKDGHEVRTCKVADKTGSINISVWDDVGNLIQPGDIIRLTKGYASVFKGCLTLYTGRGGDLQKIGEFCMVYSEVPNFSEPNPEYSTQQAPNKAVQNDSNPSASQPTTGPSAASPASENQNGNGLSAPPGPGGGPHPPHTPSHPPSTRITRSQPNHTPAGPPGPSSNPVSNGKETRRSSKR

SEQ ID NO: 3 - protein, 114 aa, Homo sapiens
  VARIANT (1)..(1): Xaa is selected from Phe, Leu and Pro
XXXDXKXGXKNXXXXFIVLEXGXXTXTKXXXEVRXXXVXDXXXXIXXSXWDXXGXXIXXGDIXRLTXGYASXXXXCLTLYXGXXGXXXKIGEXCMVXXEXXNXSEPXXXXXXQX

SEQ ID NO: 4 - protein, 204 aa, Homo sapiens
MNRVNDPLIFIRDIKPGLKNLNVVFIVLEIGRVTKTKDGHEVRSCKVADKTGSITISVWDEIGGLIQPGDIIRLTRGYASMWKGCLTLYTGRGGELQKIGEFCMVYSEVPNFSEPNPDYRGQQNKGAQSEQKNNSMNSNMGTGTFGPVGNGVHTGPESREHQFSHAGRSNGRGLINPQLQGTASNQTVMTTISNGRDPRRAFKR

SEQ ID NO: 5 - protein, 11 aa, Homo sapiens
NPEYSTQQAPN

SEQ ID NO: 6 - DNA, 636 bp, Homo sapiens
atgacgacggagacctttgtgaaggatatcaagcctgggctcaagaatctgaaccttatcttcattgtgctggagacaggccgagtgaccaagacaaaggacgggcatgaggttcggacctgcaaagtggcggacaaaacaggcagcatcaatatctctgtctgggacgatgttggcaatctgatccagcctggggacattatccggctcaccaaagggtacgcttcagttttcaaaggttgtctgacactatatactggccgtgggggtgatctgcagaagattggagaattctgtatggtttattctgaggttcctaacttcagtgagccaaacccagagtacagcacccagcaggcacccaacaaggcggtgcagaacgacagcaacccttcagcttcccagcctaccactggaccctctgctgcctctccagcctctgagaaccagaatgggaatggactgagtgccccaccaggtcccggtggtggcccacatccccctcatactccctcccacccacccagcacccgaatcactcgaagccagcccaaccacacacctgcaggcccgcctggcccttccagcaaccctgttagtaacggcaaagaaacccggaggagcagcaagagatag

SEQ ID NO: 7 - DNA, 615 bp, Homo sapiens
atgaatagggtcaacgacccacttatttttataagagatattaagcccggactgaaaaacttaaatgtcgtctttattgtcctggagataggacgcgtgaccaaaaccaaagacggccatgaagtgagatcgtgcaaagtagcagataaaacgggcagcatcactatttccgtgtgggatgagatcggaggtcttatacagccaggggatattattcggttgaccagagggtatgcatccatgtggaaaggatgtctgacactttatactggaaggggtggtgaacttcaaaaaattggggaattttgtatggtttattcagaagtgccaaatttcagtgaacccaacccagattatcgaggacagcagaacaaaggggcacagagtgaacagaagaataattccatgaatagtaatatgggtacaggtacatttggaccagtgggaaatggtgttcacactggccctgaatcaagggaacaccagttttcacatgctggcagaagcaatggccggggacttataaatccacaactacaaggaacagctagtaatcaaacagtgatgaccacaataagtaatggcagggaccctcggagagcctttaaaagatga

SEQ ID NO: 8 - DNA, 21 bp, Homo sapiens
gacaaaggacgggcatgagtt

SEQ ID NO: 9 - DNA, 21 bp, Homo sapiens
ttcuguuuccugcccguacuc

SEQ ID NO: 10 - protein, 114 aa, Homo sapiens
  VARIANT (2)..(2): Xaa is selected from Val and Ile
FXXDXKXGLKNLNXXFIVLEXGRVTKTKDGHEVRXCKVADXTGSIXISVWDXXGXLIQXGDIIRLTXGYASXXKGCLTLYTGRGGXLQKIGEFCMVYSEVPNFSEPNPXYXXQQ

SEQ ID NO: 11 - DNA, 19 bp, Homo sapiens
gcgcctgattcgagatccu

SEQ ID NO: 12 - DNA, 19 bp, Homo sapiens
uucuccgaacgugucacgu

SEQ ID NO: 13 - protein, 206 aa, Homo sapiens
MTTETFVKDIKPGLKNLNLIFIVLETGRVTKTKDGHEVRTCKVADKTGSINISVWDDVGNLIQPGDIIRLTKGYASVFKGCLTLYTGRGGDLQKIGEFCMVYSEVPNFSEPNPEYSTQQAPNKAVQNDSNPSASQPTTGPSAASPASENQNGNGLSAPPGPGGGNPEYSTQQAPNRSQPNHTPAGPPGPSSNPVSNGKETRRSSKR

SEQ ID NO: 14 - protein, 212 aa, Mus musculus
MTTETFVKDIKPGLKNLNLIFIVLETGRVTKTKDGHEVRTCKVADKTGSINISVWDDVGNLIQPGDIIRLTKGYASVFKGCLTLYTGRGGDLQKIGEFCMVYSEVPNFSEPNPEYNTQQAPNKSVQNNDNSPTAPQATTGPPAASPASENQNGNGLSTQLGPVGGPHPSHTPSHPPSTRITRSQPNHTPSGPPGPSSNPVSNGKETRRSSKR

SEQ ID NO: 15 - protein, 203 aa, Xenopus laevis
MTTETFVKDVKPGLKNLSVLFIVLETGRVTKTKDGHEVRTCKVADKTGSINISVWDDLGNFIQPGDIIRLTKGYASLFKGCLTLYTGRGGDLQKIGEFCMVYSEVPNFSEPNPEYIAQQSQNKQAQAESGTGTNSHNSSSPAPPASDLENGNGSNSSGPPTHQSTAPTHSTSGRITRSQPNHSIPGAPNSVSNGKEPRRTGKR

SEQ ID NO: 16 - protein, 204 aa, Homo sapiens; identical to SEQ ID NO: 4

SEQ ID NO: 17 - protein, 198 aa, Mus musculus
MHGVNDPPLFIKDIKAGLKNLNVVFIVLEIGRVTKTKDGHEVRSCKVADRTGSITISVWDEIGGLIQTGDIIRLTRGYASMWKGCLTLYTGRGGELQKIGEFCMVYSEVPNFSEPNPDYRGQQNRGVQNEQKDKLSTNTFGPVGNGDQTGPESRGYHLPYGRSNGPGPISPQLPGTPSSQTVRTTISNARDPRRAFKR

SEQ ID NO: 18 - protein, 211 aa, Danio rerio
MSNISNEAVILIKDVKPGSKNLNIVFIVLEIGRVTKTKDGHEVRSCRVADKSGSIAISVWDELGSLIQPGDIIRLTRGYASIWKGCLTLYTGRGGDLQKIGEFCMVYSEVPNFSEPNPELLAQANQQNKTSKEQRGNSPPNQNAGNGTVPVFSNNNAAPVPRDPNFGASGRPNGRAPGNGPPPVTAGGTPAPPKPTVSISNGRDPRRASKR

SEQ ID NO: 19 - protein, 204 aa, Drosophila melanogaster
MYNVECIPIKDIKPGLKNINVIFIVLEVGVATVTKENREVRNFKVGDPTACINVSIWDEPGKLIAPGDIVRLTKGYASIWRHCLTLYSGKNGEVFKIGEYCMVFNESVNMSEPKRAEQQAVANPAATPAGLPAGGGAPGLPAKGGATGIPQPAVAAAPGAPATQSAVTTAPAAAPAIAPQTTTKPGTRGGRGGGGRGGLKGERR

SEQ ID NO: 20 - protein, 154 aa, Sulfolobus solfataricus
MEEKVGNLKPNMNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE